SEQUENCE LISTING (68 sequences)

SEQ ID NO: 1 - RNA, 15 nt, synthetic construct
  source 1..15: mol_type = other RNA
  modified_base 15: mod_base = OTHER; note = 3'-Triethylene glycol cholesterol modified nucleotide
taactgtgacagtga

SEQ ID NO: 2 - RNA, 20 nt, synthetic construct
  source 1..20: mol_type = other RNA
tcactgtcacagttagtgtg

SEQ ID NO: 3 - RNA, 15 nt, synthetic construct
  source 1..15: mol_type = other RNA
  modified_base 15: mod_base = OTHER; note = 3'-Triethylene glycol cholesterol modified nucleotide
aactgtgacagtgca

SEQ ID NO: 4 - RNA, 20 nt, synthetic construct
  source 1..20: mol_type = other RNA
tgcactgtcacagttagtgt

SEQ ID NO: 5 - RNA, 15 nt, synthetic construct
  source 1..15: mol_type = other RNA
  modified_base 15: mod_base = OTHER; note = 3'-Triethylene glycol cholesterol modified nucleotide
actgtgacagtgcca

SEQ ID NO: 6 - RNA, 20 nt, synthetic construct
  source 1..20: mol_type = other RNA
tggcactgtcacagttagtg

SEQ ID NO: 7 - RNA, 15 nt, synthetic construct
  source 1..15: mol_type = other RNA
  modified_base 15: mod_base = OTHER; note = 3'-Triethylene glycol cholesterol modified nucleotide
ctgtgacagtgccta

SEQ ID NO: 8 - RNA, 20 nt, synthetic construct
  source 1..20: mol_type = other RNA
taggcactgtcacagttagt

SEQ ID NO: 9 - RNA, 15 nt, synthetic construct
  source 1..15: mol_type = other RNA
  modified_base 15: mod_base = OTHER; note = 3'-Triethylene glycol cholesterol modified nucleotide
tgtgacagtgcctga

SEQ ID NO: 10 - RNA, 20 nt, synthetic construct
  source 1..20: mol_type = other RNA
tcaggcactgtcacagttag

SEQ ID NO: 11 - RNA, 15 nt, synthetic construct
  source 1..15: mol_type = other RNA
  modified_base 15: mod_base = OTHER; note = 3'-Triethylene glycol cholesterol modified nucleotide
gtgacagtgcctgga

SEQ ID NO: 12 - RNA, 20 nt, synthetic construct
  source 1..20: mol_type = other RNA
tccaggcactgtcacagtta

SEQ ID NO: 13 - RNA, 15 nt, synthetic construct
  source 1..15: mol_type = other RNA
  modified_base 15: mod_base = OTHER; note = 3'-Triethylene glycol cholesterol modified nucleotide
tgacagtgcctggaa

SEQ ID NO: 14 - RNA, 20 nt, synthetic construct
  source 1..20: mol_type = other RNA
ttccaggcactgtcacagtt

SEQ ID NO: 15 - RNA, 15 nt, synthetic construct
  source 1..15: mol_type = other RNA
  modified_base 15: mod_base = OTHER; note = 3'-Triethylene glycol cholesterol modified nucleotide
gacagtgcctggaga

SEQ ID NO: 16 - RNA, 20 nt, synthetic construct
  source 1..20: mol_type = other RNA
tctccaggcactgtcacagt

SEQ ID NO: 17 - RNA, 15 nt, synthetic construct
  source 1..15: mol_type = other RNA
  modified_base 15: mod_base = OTHER; note = 3'-Triethylene glycol cholesterol modified nucleotide
acagtgcctggagga

SEQ ID NO: 18 - RNA, 20 nt, synthetic construct
  source 1..20: mol_type = other RNA
tcctccaggcactgtcacag

SEQ ID NO: 19 - RNA, 15 nt, synthetic construct
  source 1..15: mol_type = other RNA
  modified_base 15: mod_base = OTHER; note = 3'-Triethylene glycol cholesterol modified nucleotide
cagtgcctggaggta

SEQ ID NO: 20 - RNA, 20 nt, synthetic construct
  source 1..20: mol_type = other RNA
tacctccaggcactgtcaca

SEQ ID NO: 21 - RNA, 15 nt, synthetic construct
  source 1..15: mol_type = other RNA
  modified_base 15: mod_base = OTHER; note = 3'-Triethylene glycol cholesterol modified nucleotide
agtgcctggaggtga

SEQ ID NO: 22 - RNA, 20 nt, synthetic construct
  source 1..20: mol_type = other RNA
tcacctccaggcactgtcac

SEQ ID NO: 23 - RNA, 15 nt, synthetic construct
  source 1..15: mol_type = other RNA
  modified_base 15: mod_base = OTHER; note = 3'-Triethylene glycol cholesterol modified nucleotide
gtgcctggaggtgaa

SEQ ID NO: 24 - RNA, 20 nt, synthetic construct
  source 1..20: mol_type = other RNA
ttcacctccaggcactgtca

SEQ ID NO: 25 - RNA, 15 nt, synthetic construct
  source 1..15: mol_type = other RNA
  modified_base 15: mod_base = OTHER; note = 3'-Triethylene glycol cholesterol modified nucleotide
tgcctggaggtgaaa

SEQ ID NO: 26 - RNA, 20 nt, synthetic construct
  source 1..20: mol_type = other RNA
tttcacctccaggcactgtc

SEQ ID NO: 27 - RNA, 14 nt, synthetic construct
  source 1..14: mol_type = other RNA
  modified_base 14: mod_base = OTHER; note = 3'-Triethylene glycol cholesterol modified nucleotide
gcctggagggaaaa

SEQ ID NO: 28 - RNA, 20 nt, synthetic construct
  source 1..20: mol_type = other RNA
ttttcacctccaggcactgt

SEQ ID NO: 29 - RNA, 15 nt, synthetic construct
  source 1..15: mol_type = other RNA
  modified_base 15: mod_base = OTHER; note = 3'-Triethylene glycol cholesterol modified nucleotide
cctggaggtgaaaaa

SEQ ID NO: 30 - RNA, 20 nt, synthetic construct
  source 1..20: mol_type = other RNA
tttttcacctccaggcactg

SEQ ID NO: 31 - RNA, 47 nt, synthetic construct
  source 1..47: mol_type = other RNA
tgtacagccacactaactgtgacagtgcctggaggtgaaaagaaagt

SEQ ID NO: 32 - RNA, 20 nt, synthetic construct
  source 1..20: mol_type = other RNA
tgacagtgcctggaggtgaa

SEQ ID NO: 33 - DNA, 433 bp, Homo sapiens
  source 1..433: mol_type = genomic DNA
gtttaaactccagccttgaaatcctgagaacccaggtggttgactgcggcgagtatacatgcaaagcttccaatgagtatggcagtgtcagctgtacagccacactaactgtgacagaggcatatccaccaacctttctctccagacctaagtcccttacaacttttgtgggtaaggcagccaagttcatctgcacagtgacagggactcctgtgatgatgtgaaggagaaaatattccaacttaaagctattccaaagaagaaagttcctgaaaaacctcaggttccagaaaaagtggagcttacacctctgaaagtgcctggaggtgaaaagaaagttcgcaaattacttccggaacgtaaacctgaaccaaaggaagaagttgttctgaaaagcgttctaagaaaaagacctgaagaagaagaactcgag

SEQ ID NO: 34 - DNA, 233 bp, Homo sapiens
  source 1..233: mol_type = genomic DNA
gtttaaactccagccttgaaatcctgagaacccaggtggttgactgcggcgagtatacatgcaaagcttccaatgagtatggcagtgtcagctgtacagccacactaactgtgacagtgcctggaggtgaaaagaaagttcgcaaattacttccggaacgtaaacctgaaccaaaggaagaagttgttctgaaaagcgttctaagaaaaagacctgaagaagaagaactcgag

SEQ ID NO: 35-36 - skipped (2 entries)

SEQ ID NO: 37 - RNA, 20 nt, synthetic construct
  source 1..20: mol_type = other RNA
  modified_base 1: mod_base = OTHER; note = 5'-phosphate-2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide with phosphorothioate linkage
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  modified_base 20: mod_base = OTHER; note = 2'-fluoro modified nucleotide
tcactgtcacagttagtgtg

SEQ ID NO: 38 - RNA, 20 nt, synthetic construct
  source 1..20: mol_type = other RNA
  modified_base 1: mod_base = OTHER; note = 5'-phosphate-2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide with phosphorothioate linkage
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  modified_base 20: mod_base = OTHER; note = 2'-fluoro modified nucleotide
tgcactgtcacagttagtgt

SEQ ID NO: 39 - RNA, 20 nt, synthetic construct
  source 1..20: mol_type = other RNA
  modified_base 1: mod_base = OTHER; note = 5'-phosphate-2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide with phosphorothioate linkage
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  modified_base 20: mod_base = OTHER; note = 2'-fluoro modified nucleotide
tggcactgtcacagttagtg

SEQ ID NO: 40 - RNA, 20 nt, synthetic construct
  source 1..20: mol_type = other RNA
  modified_base 1: mod_base = OTHER; note = 5'-phosphate-2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide with phosphorothioate linkage
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  modified_base 20: mod_base = OTHER; note = 2'-fluoro modified nucleotide
taggcactgtcacagttagt

SEQ ID NO: 41 - RNA, 20 nt, synthetic construct
  source 1..20: mol_type = other RNA
  modified_base 1: mod_base = OTHER; note = 5'-phosphate-2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide with phosphorothioate linkage
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  modified_base 20: mod_base = OTHER; note = 2'-fluoro modified nucleotide
tcaggcactgtcacagttag

SEQ ID NO: 42 - RNA, 20 nt, synthetic construct
  source 1..20: mol_type = other RNA
  modified_base 1: mod_base = OTHER; note = 5'-phosphate-2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide with phosphorothioate linkage
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  modified_base 20: mod_base = OTHER; note = 2'-fluoro modified nucleotide
tccaggcactgtcacagtta

SEQ ID NO: 43 - RNA, 20 nt, synthetic construct
  source 1..20: mol_type = other RNA
  modified_base 1: mod_base = OTHER; note = 5'-phosphate-2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide with phosphorothioate linkage
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  modified_base 20: mod_base = OTHER; note = 2'-fluoro modified nucleotide
ttccaggcactgtcacagtt

SEQ ID NO: 44 - RNA, 20 nt, synthetic construct
  source 1..20: mol_type = other RNA
  modified_base 1: mod_base = OTHER; note = 5'-phosphate-2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide with phosphorothioate linkage
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  modified_base 20: mod_base = OTHER; note = 2'-fluoro modified nucleotide
tcctccaggcactgtcacag

SEQ ID NO: 45 - RNA, 20 nt, synthetic construct
  source 1..20: mol_type = other RNA
  modified_base 1: mod_base = OTHER; note = 5'-phosphate-2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide with phosphorothioate linkage
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  modified_base 20: mod_base = OTHER; note = 2'-fluoro modified nucleotide
tacctccaggcactgtcaca

SEQ ID NO: 46 - RNA, 20 nt, synthetic construct
  source 1..20: mol_type = other RNA
  modified_base 1: mod_base = OTHER; note = 5'-phosphate-2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide with phosphorothioate linkage
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  modified_base 20: mod_base = OTHER; note = 2'-fluoro modified nucleotide
tcacctccaggcactgtcac

SEQ ID NO: 47 - RNA, 20 nt, synthetic construct
  source 1..20: mol_type = other RNA
  modified_base 1: mod_base = OTHER; note = 5'-phosphate-2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide with phosphorothioate linkage
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  modified_base 20: mod_base = OTHER; note = 2'-fluoro modified nucleotide
ttcacctccaggcactgtca

SEQ ID NO: 48 - RNA, 20 nt, synthetic construct
  source 1..20: mol_type = other RNA
  modified_base 1: mod_base = OTHER; note = 5'-phosphate-2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide with phosphorothioate linkage
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  modified_base 20: mod_base = OTHER; note = 2'-fluoro modified nucleotide
tttcacctccaggcactgtc

SEQ ID NO: 49 - RNA, 20 nt, synthetic construct
  source 1..20: mol_type = other RNA
  modified_base 1: mod_base = OTHER; note = 5'-phosphate-2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide with phosphorothioate linkage
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  modified_base 20: mod_base = OTHER; note = 2'-fluoro modified nucleotide
ttttcacctccaggcactgt

SEQ ID NO: 50 - RNA, 20 nt, synthetic construct
  source 1..20: mol_type = other RNA
  modified_base 1: mod_base = OTHER; note = 5'-phosphate-2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide with phosphorothioate linkage
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  modified_base 20: mod_base = OTHER; note = 2'-fluoro modified nucleotide
tttttcacctccaggcactg

SEQ ID NO: 51 - DNA, 46 bp, Homo sapiens
  source 1..46: mol_type = genomic DNA
gtacagccacactaactgtgacagtgcctggaggtgaaaagaaagt

SEQ ID NO: 52 - DNA, 48 bp, Homo sapiens
  source 1..48: mol_type = genomic DNA
tgtacagccacactaactgtgacagtgcctggaggtgaaaagaaagtt

SEQ ID NO: 53 - protein, 16 aa, Homo sapiens
CTATLTVTVPGGEKKV

SEQ ID NO: 54 - RNA, 15 nt, synthetic construct
  source 1..15: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide with phosphorothioate linkage
  modified_base 15: mod_base = OTHER; note = 2'-fluoro-3'-Triethylene glycol cholesterol modified nucleotide with phosphorothioate linkage
taactgtgacagtga

SEQ ID NO: 55 - RNA, 15 nt, synthetic construct
  source 1..15: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide with phosphorothioate linkage
  modified_base 15: mod_base = OTHER; note = 2'-fluoro-3'-Triethylene glycol cholesterol modified nucleotide with phosphorothioate linkage
aactgtgacagtgca

SEQ ID NO: 56 - RNA, 15 nt, synthetic construct
  source 1..15: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide with phosphorothioate linkage
  modified_base 15: mod_base = OTHER; note = 2'-fluoro-3'-Triethylene glycol cholesterol modified nucleotide with phosphorothioate linkage
actgtgacagtgcca

SEQ ID NO: 57 - RNA, 15 nt, synthetic construct
  source 1..15: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide with phosphorothioate linkage
  modified_base 15: mod_base = OTHER; note = 2'-fluoro-3'-Triethylene glycol cholesterol modified nucleotide with phosphorothioate linkage
ctgtgacagtgccta

SEQ ID NO: 58 - RNA, 15 nt, synthetic construct
  source 1..15: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide with phosphorothioate linkage
  modified_base 15: mod_base = OTHER; note = 2'-fluoro-3'-Triethylene glycol cholesterol modified nucleotide with phosphorothioate linkage
tgtgacagtgcctga

SEQ ID NO: 59 - RNA, 15 nt, synthetic construct
  source 1..15: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide with phosphorothioate linkage
  modified_base 15: mod_base = OTHER; note = 2'-fluoro-3'-Triethylene glycol cholesterol modified nucleotide with phosphorothioate linkage
gtgacagtgcctgga

SEQ ID NO: 60 - RNA, 15 nt, synthetic construct
  source 1..15: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide with phosphorothioate linkage
  modified_base 15: mod_base = OTHER; note = 2'-fluoro-3'-Triethylene glycol cholesterol modified nucleotide with phosphorothioate linkage
tgacagtgcctggaa

SEQ ID NO: 61 - RNA, 15 nt, synthetic construct
  source 1..15: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide with phosphorothioate linkage
  modified_base 15: mod_base = OTHER; note = 2'-fluoro-3'-Triethylene glycol cholesterol modified nucleotide with phosphorothioate linkage
gacagtgcctggaga

SEQ ID NO: 62 - RNA, 15 nt, synthetic construct
  source 1..15: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide with phosphorothioate linkage
  modified_base 15: mod_base = OTHER; note = 2'-fluoro-3'-Triethylene glycol cholesterol modified nucleotide with phosphorothioate linkage
acagtgcctggagga

SEQ ID NO: 63 - RNA, 15 nt, synthetic construct
  source 1..15: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide with phosphorothioate linkage
  modified_base 15: mod_base = OTHER; note = 2'-fluoro-3'-Triethylene glycol cholesterol modified nucleotide with phosphorothioate linkage
cagtgcctggaggta

SEQ ID NO: 64 - RNA, 15 nt, synthetic construct
  source 1..15: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide with phosphorothioate linkage
  modified_base 15: mod_base = OTHER; note = 2'-fluoro-3'-Triethylene glycol cholesterol modified nucleotide with phosphorothioate linkage
agtgcctggaggtga

SEQ ID NO: 65 - RNA, 15 nt, synthetic construct
  source 1..15: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide with phosphorothioate linkage
  modified_base 15: mod_base = OTHER; note = 2'-fluoro-3'-Triethylene glycol cholesterol modified nucleotide with phosphorothioate linkage
gtgcctggaggtgaa

SEQ ID NO: 66 - RNA, 15 nt, synthetic construct
  source 1..15: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide with phosphorothioate linkage
  modified_base 15: mod_base = OTHER; note = 2'-fluoro-3'-Triethylene glycol cholesterol modified nucleotide with phosphorothioate linkage
tgcctggaggtgaaa

SEQ ID NO: 67 - RNA, 15 nt, synthetic construct
  source 1..15: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide with phosphorothioate linkage
  modified_base 15: mod_base = OTHER; note = 2'-fluoro-3'-Triethylene glycol cholesterol modified nucleotide with phosphorothioate linkage
gcctggaggtgaaaa

SEQ ID NO: 68 - RNA, 15 nt, synthetic construct
  source 1..15: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide with phosphorothioate linkage
  modified_base 15: mod_base = OTHER; note = 2'-fluoro-3'-Triethylene glycol cholesterol modified nucleotide with phosphorothioate linkage
cctggaggtgaaaaa

(19) United States
(12) Patent Application Publication
(10) Pub. No.: US 2026/0242787 A1
(43) Pub. Date: Aug. 20, 2026

(54) Methods and Compositions for Preventing, Treating, or Reversing Cardiac Diastolic Dysfunction

(71) Applicant: GenKardia Inc., Dover, DE (US)

(72) Inventor: David C. Zebrowski, Dover, DE (US)

(21) Appl. No.: 19/130,953

(22) PCT Filed: Nov. 20, 2023
(86) PCT No.: PCT/US2023/080551

§ 371 Date: May 18, 2025

Related U.S. Application Data
(60) Provisional application No. 63/384,389, filed on Nov. 18, 2022.

(51) Int. Cl. C12N 15/113; A61P 9/00

(52) U.S. Cl. CPC C12N 15/113; A61P 9/00; C12N 2310/11

(57) ABSTRACT

The present disclosure provides methods and compositions for treating or preventing diastolic dysfunction and/or diastolic heart failure in a human subject, comprising administering to the subject an oligonucleotide that suppresses the expression of the Titin isoform N2B. Also provided are additional methods and oligonucleotides that may be used as described herein.

### 1. SEQUENCE LISTING

The instant application contains a Sequence Listing with 68 sequences, which has been submitted via Patent Center and is hereby incorporated by reference in its entirety. Said XML copy, created on Nov. 3, 2023, is named 51419WO_CRF_sequencelisting, and is 349 KB in size.

### 2. BACKGROUND

Heart disease is currently the leading cause of death in the industrialized world. Heart disease can lead to heart failure. Heart failure is a clinical condition that describes the inability of the heart to adequately deliver requisite levels of oxygenated blood to the body to maintain normal tissue and organ function.

The left ventricular cardiac cycle (henceforth referred to as cardiac cycle) refers to the filling and ejection of oxygenated blood. The cardiac cycle is divided into Diastole and Systole. Diastole refers to the phase of the cardiac cycle when the ventricle of the heart relaxes and fills with incoming oxygenated blood from the lungs. Systole refers to the phase of the cardiac cycle when the ventricle contracts and ejects oxygenated blood throughout the vasculature to tissues and organs of the body. Heart failure can result from a dysfunction during Diastole or Systole.

Diastolic dysfunction represents an inability of the left ventricle of the heart to properly relax and fill with oxygenated blood. As a result, when the left ventricle of the heart contracts, an inadequate amount of oxygenated blood is dispersed through the vasculature to the tissue and organs of the body. Systolic dysfunction represents an inability of the left ventricle of the heart to properly contract. As a result, when the left ventricle of the heart contracts, an inadequate amount of oxygenated blood is dispersed through the vasculature to the tissue and organs of the body.

Approximately half of patients diagnosed with heart failure have Diastolic dysfunction, i.e., have Diastolic Heart Failure (DHF). For instance, patients with (i) Heart Failure with preserved Ejection Fraction (HFpEF), (ii) cardiomyopathies such as Restrictive Cardiomyopathy (RCM), (iii) obstructive and non-obstructive hypertrophic Cardiomyopathy (HCM), (iv) infiltrative cardiomyopathies (e.g., Amyloidosis), and (v) Chagas disease exhibit DHF. Despite the prevalence of DHF, there is currently no treatment. Identifying methods to promote cardiac relaxation presents a method to improve cardiac function and treat patients that suffer from DHF.

### 3. SUMMARY

The present disclosure provides oligonucleotides, pharmaceutical compositions comprising the oligonucleotides, methods for modulating the expression of Titin (TTN) isoform N2B (and Titin isoforms Novex-1, and Novex-2), and the use of these in treating diseases, such as cardiac disorders (e.g., cardiac disorders associated with diastolic dysfunction). In certain embodiments, this disclosure relates to oligonucleotides that are hybridizable with nucleic acid sequences present in mRNA of Titin isoform N2B and not present in mRNA of Titin isoform N2BA, for use in medicine, particularly in the treatment or prevention of cardiac disorders, particularly in the treatment or prevention of disorders associated with cardiac diastolic dysfunction, such as, for example Heart Failure with preserved Ejection Fraction (HFpEF), restrictive cardiomyopathy (RCM), hypertrophic cardiomyopathy (HCM), and/or infiltrative cardiomyopathies.

The present disclosure utilizes an exon 50-exon 220 boundary sequence present in Titin isoform N2B but not present in Titin isoform N2BA, such as, for example SEQ ID NOs: 31 or 32, to achieve inhibition of Titin isoform N2B via a hybridizable oligonucleotide (e.g., siRNA), increase the ratio of Titin isoform N2BA to Titin isoform N2B, and improve diastolic function in patients with diastolic heart failure (DHF).

In one aspect, this disclosure features a method of treating or preventing diastolic dysfunction in a human subject, comprising administering to the subject an oligonucleotide that suppresses the expression of the Titin isoform N2B.

In another aspect, this disclosure features a method of treating or preventing diastolic heart failure in a human subject, comprising administering to the subject an oligonucleotide that suppresses the expression of the Titin isoform N2B.

In another aspect, this disclosure features a method of treating or preventing diastolic dysfunction in a human subject, comprising administering an oligonucleotide to the subject, wherein the oligonucleotide is substantially complementary to an exon-exon boundary sequence formed by splicing of two non-sequential exons present in the TTN pre-mRNA transcript, and wherein the exon-exon boundary sequence is present in the N2B mRNA transcript but not the N2BA mRNA transcript.

In another aspect, this disclosure features a method of treating or preventing diastolic heart failure in a human subject, comprising administering an oligonucleotide to the subject, wherein the oligonucleotide is substantially complementary to an exon-exon boundary sequence formed by splicing of two non-sequential exons present in the TTN pre-mRNA transcript, and wherein the exon-exon boundary sequence is present in the N2B mRNA transcript but not the N2BA mRNA transcript.

In another aspect, this disclosure features a method of treating or preventing diastolic dysfunction in a patient in need thereof, comprising administering to the patient an oligonucleotide that specifically hybridizes with the nucleic acid of the Titin isoform N2B mature mRNA transcript to a greater extent than with the nucleic acid of the Titin isoform N2BA mature mRNA transcript.

In another aspect, this disclosure features a method of treating diastolic heart failure in a patient in need thereof, comprising administering to the patient an oligonucleotide that specifically hybridizes with the RNA nucleic acid of the Titin isoform N2B mature mRNA transcript to a greater extent than with the RNA nucleic acid of the Titin isoform N2BA mature mRNA transcript.

In some embodiments, the oligonucleotide is double-stranded.

In some embodiments, the oligonucleotide is a small interfering RNA (siRNA), a short hairpin RNA (shRNA), or a micro RNA (miRNA).

In some embodiments, the oligonucleotide comprises a small interfering RNA (siRNA).

In some embodiments, the siRNA comprises (a) a duplex region and (b) optionally one or two overhang regions, wherein each overhang region is six or fewer nucleotides, wherein the duplex region comprises a sense and an antisense region each comprising between 15 and 30 nucleotides, and wherein the antisense region comprises a nucleotide sequence that is substantially complementary to SEQ ID NOs: 31 or 32.

In some embodiments, the antisense region comprises a nucleotide sequence that is substantially complementary to a contiguous sequence of at least 18 nucleotides of SEQ ID NO: 31. In some embodiments, the antisense region is 85% complementary to a contiguous sequence of at least 18 nucleotides of SEQ ID NO: 31. In some embodiments, the antisense region is 90% complementary to a contiguous sequence of at least 18 nucleotides of SEQ ID NO: 31. In some embodiments, the antisense region is 95% complementary to a contiguous sequence of at least 18 nucleotides of SEQ ID NO: 31. In some embodiments, the antisense region is 100% complementary to a contiguous sequence of at least 18 nucleotides of SEQ ID NO: 31.

In some embodiments, the contiguous sequence comprises positions 7 to 24 of SEQ ID NO: 31. In some embodiments, the contiguous sequence comprises positions 6 to 23 of SEQ ID NO: 31. In some embodiments, the contiguous sequence comprises positions 8 to 25 of SEQ ID NO: 31.

In some embodiments, the antisense region comprises a nucleotide sequence that is substantially complementary to SEQ ID NO: 32.

In some embodiments, the antisense region is at least 85% complementary to SEQ ID NO: 32. In some embodiments, the antisense region is at least 90% complementary to SEQ ID NO: 32. In some embodiments, the antisense region is at least 95% complementary to SEQ ID NO: 32. In some embodiments, the antisense region is 100% complementary to SEQ ID NO: 32.

In some embodiments, the sense and antisense regions each comprise between 13 and 25 nucleotides.

In some embodiments, the sense and antisense regions each comprise between 15 and 22 nucleotides.

In some embodiments, the antisense sequence comprises at least 85% sequence identity to a sequence selected from SEQ ID NOs: 2, 4, 6, 8, 10, 12, 14, 22, 24, 26, 28, and 30. In some embodiments, the antisense region has at least 90% sequence identity to a sequence selected from SEQ ID NOs: 2, 4, 6, 8, 10, 12, 14, 22, 24, 26, 28, and 30. In some embodiments, the antisense region has at least 95% sequence identity to a sequence selected from SEQ ID NOs: 2, 4, 6, 8, 10, 12, 14, 22, 24, 26, 28, and 30. In some embodiments, the antisense region has a sequence selected from SEQ ID NOs: 2, 4, 6, 8, 10, 12, 14, 22, 24, 26, 28, and 30. In some embodiments, the the antisense region has a sequence selected from SEQ ID NOs: 22, 24, 26, 28, and 30. In some embodiments, the antisense region has a sequence selected from SEQ ID NOS: 22, 24, and 26. In some embodiments, the antisense region is SEQ ID NO: 22. In some embodiments, the antisense region is SEQ ID NO: 26. In particular embodiments, the antisense region is SEQ ID NO: 24.

In some embodiments, the sense region, the antisense region, or both the sense and antisense regions are chemically modified.

In some embodiments, the chemical modifications are selected from modifications at the 2′-position of the ribose sugar of one or more nucleotides of the sense and antisense regions, substitutions of nucleotides with other nucleotides, non-natural phosphodiester linkages, or combinations thereof.

In some embodiments, at least a portion of the chemical modifications are at the 2′-position of the ribose sugar of one or more nucleotides of the sense region, antisense region, or both the sense and antisense regions.

In some embodiments, at least 5 of the nucleotides of the sense and antisense regions are modified at the 2′position of the ribose sugars of the nucleotides. In some embodiments, at least 10 of the nucleotides of the sense and antisense regions are modified at the 2′position of the ribose sugars of the nucleotides. In some embodiments, the modifications at the 2′-position comprise replacing the OH group with one or more groups selected from H, halo, R, OR, SH, SR, NH₂, NHR, and NRR′, wherein R and R′ are independently C₁₋₆alkyl.

In some embodiments, the sense region, antisense region, or both the sense and antisense regions comprise at least one non-natural phosphodiester linkage.

In some embodiments, the sense region, antisense region, or both the sense and antisense regions comprise at least five non-natural phosphodiester linkages.

In some embodiments, the non-natural phosphodiester linkages are selected from phosphorothioate, methylphosphonate, and a peptide.

In some embodiments, the chemical modification comprises substitution of least one uracil nucleotide in the sense or antisense region with a 5-propynyluracil nucleotide.

In some embodiments, the chemical modification comprises substitution of least one uracil nucleotide in the sense or antisense region with a 5-methyluridine nucleotide.

In some embodiments, the chemical modification comprises substitution of least one uracil nucleotide in the sense or antisense region with a deoxythymidine nucleotide.

In some embodiments, the chemical modification comprises substitution of least one cytosine nucleotide in the sense or antisense region with a 5-methylcytosine nucleotide.

In some embodiments, the chemical modification comprises substitution of least one adenine or guanine nucleotide in the sense or antisense region with an 8-bromoadenine or 8-bromoguanine nucleotide.

In some embodiments, the siRNA is conjugated to a non-nucleic acid molecule, wherein the non-nucleic acid molecule comprises a fatty acid, lipid, peptide, protein, antibody, or nanoparticle.

In some embodiments, the siRNA is conjugated to cholesterol.

In some embodiments, the administering of the oligonucleotide suppresses the expression of a Titin isoform N2BA to a lesser extent than the oligonucleotide suppresses the expression of a Titin isoform N2B.

In some embodiments, the oligonucleotide suppresses the expression of the Titin isoform N2B from about 2-fold to about 1000-fold greater than the oligonucleotide suppresses expression of the Titin isoform N2BA.

In some embodiments, the administering of the oligonucleotide inhibits the expression of the Titin isoform N2BA by 10% or less.

In some embodiments, the oligonucleotide increases the ratio of Titin isoform N2BA to Titin isoform N2B in the cardiomyocytes of the subject.

In some embodiments, the ratio of the Titin isoform N2BA to the Titin isoform N2B is increased from about 1.2-fold to about 5-fold.

In some embodiments, the exon-exon boundary sequence is formed from splicing TTN exons 50 and 220.

In some embodiments, the exon-exon boundary sequence is formed from splicing TTN exons 10 and 12.

In some embodiments, the length of the exon-exon boundary sequence is from about 15 nucleotides to 50 nucleotides. In some embodiments, the length of the exon-exon boundary sequence is from about 15 nucleotides to 30 nucleotides. In some embodiments, the length of the exon-exon boundary sequence is from about 15 nucleotides to 27 nucleotides. In some embodiments, the length of the exon-exon boundary sequence is from about 20 nucleotides to 27 nucleotides. The method of any one of claims 1-56, wherein the oligonucleotide reduces expression of the N2B isoform of Titin by from about 10% to about 90%. The method of any one of claims 1-57, wherein the oligonucleotide is administered in a pharmaceutically acceptable composition.

In some embodiments, the pharmaceutically acceptable composition is a liposomal composition.

In some embodiments, the method also includes administering one or more additional medicaments selected from angiotensin-modulating agents, beta-blockers, diuretics, aldosterone antagonists, vasodilators, neprilysin inhibitors, and ionotropic agents.

In another aspect, this disclosure features an oligonucleotide that specifically hybridizes with the RNA of a Titin isoform N2B mature mRNA transcript.

In some embodiments, the oligonucleotide hybridizes with the Titin isoform N2B mature mRNA transcript to a greater extent than with a Titin isoform N2BA mature mRNA transcript.

In some embodiments, the oligonucleotide is double-stranded.

In some embodiments, the oligonucleotide comprises a small interfering RNA (siRNA), an aptamer, a short hairpin RNA (shRNA), or a micro RNA (miRNA).

In some embodiments, the oligonucleotide comprises a small interfering RNA (siRNA).

In some embodiments, the siRNA comprises (a) a duplex region and (b) optionally one or two overhang regions, wherein each overhang region is six or fewer nucleotides, wherein the duplex region comprises a sense and an antisense region each comprising between 15 and 30 nucleotides, and wherein the antisense region comprises a nucleotide sequence that is substantially complementary to SEQ ID NOs: 31 or 32.

In some embodiments, the antisense region comprises a nucleotide sequence that is substantially complementary to a contiguous sequence of at least 18 nucleotides of SEQ ID NO: 31. In some embodiments, the antisense region is 85% complementary to a contiguous sequence of at least 18 nucleotides of SEQ ID NO: 31. In some embodiments, the antisense region is 90% complementary to a contiguous sequence of at least 18 nucleotides of SEQ ID NO: 31. In some embodiments, the antisense region is 95% complementary to a contiguous sequence of at least 18 nucleotides of SEQ ID NO: 31. In some embodiments, the antisense region is 100% complementary to a contiguous sequence of at least 18 nucleotides of SEQ ID NO: 31.

In some embodiments, the contiguous sequence comprises positions 7 to 24 of SEQ ID NO: 31. In some embodiments, the contiguous sequence comprises positions 6 to 23 of SEQ ID NO: 31. In some embodiments, the contiguous sequence comprises positions 8 to 25 of SEQ ID NO: 31.

In some embodiments, the antisense region comprises a nucleotide sequence that is substantially complementary to SEQ ID NO: 32. In some embodiments, the antisense region is at least 85% complementary to SEQ ID NO: 32. In some embodiments, the antisense region is at least 90% complementary to SEQ ID NO: 32. In some embodiments, the antisense region is at least 95% complementary to SEQ ID NO: 32. In some embodiments, the antisense region is 100% complementary to SEQ ID NO: 32.

In some embodiments, the sense and antisense regions each comprise between 13 and 25 nucleotides.

In some embodiments, the sense and antisense regions each comprise between 15 and 22 nucleotides.

In some embodiments, the antisense sequence comprises at least 85% sequence identity to a sequence selected from SEQ ID NOs: 2, 4, 6, 8, 10, 12, 14, 22, 24, 26, 28, and 30. In some embodiments, the antisense region is at least 90% sequence identity to a sequence selected from SEQ ID NOs: 2, 4, 6, 8, 10, 12, 20, 22, 24, 26, 28, and 30. In some embodiments, the antisense region is at least 95% sequence identity to a sequence selected from SEQ ID NOs: 2, 4, 6, 8, 10, 12, 20, 22, 24, 26, 28, and 30. In some embodiments, the antisense region is a sequence selected from SEQ ID NOs: 2, 4, 6, 8, 10, 12, 14, 22, 24, 26, 28, and 30.

In some embodiments, the sense region, the antisense region, or both the sense and antisense regions are chemically modified.

In some embodiments, the chemical modifications are selected from modifications at the 2′-position of the ribose sugar of one or more nucleotides of the sense and antisense regions, substitutions of nucleotides with other nucleotides, non-natural phosphodiester linkages, or combinations thereof.

In some embodiments, at least a portion of the chemical modifications are at the 2′-position of the ribose sugar of one or more nucleotides of the sense region, antisense region, or both the sense and antisense regions.

In some embodiments, at least 5 of the nucleotides of the sense and antisense regions are modified at the 2′position of the ribose sugars of the nucleotides.

In some embodiments, at least 10 of the nucleotides of the sense and antisense regions are modified at the 2′position of the ribose sugars of the nucleotides.

In some embodiments, the modifications at the 2′-position comprise replacing the OH group with one or more groups selected from H, halo, R, OR, SH, SR, NH₂, NHR, and NRR′, wherein R and R′ are independently C₁₋₆alkyl.

In some embodiments, the sense region, antisense region, or both the sense and antisense regions comprise at least one non-natural phosphodiester linkage.

In some embodiments, the sense region, antisense region, or both the sense and antisense regions comprise at least five non-natural phosphodiester linkages.

In some embodiments, the non-natural phosphodiester linkages are selected from phosphorothioate, methylphosphonate, and a peptide.

In some embodiments, the chemical modification comprises substitution of least one uracil nucleotide in the sense or antisense region with a 5-propynyluracil nucleotide.

In some embodiments, the chemical modification comprises substitution of least one uracil nucleotide in the sense or antisense region with a 5-methyluridine nucleotide.

In some embodiments, the chemical modification comprises substitution of least one uracil nucleotide in the sense or antisense region with a deoxythymidine nucleotide.

In some embodiments, the chemical modification comprises substitution of least one cytosine nucleotide in the sense or antisense region with a 5-methylcytosine nucleotide.

In some embodiments, the chemical modification comprises substitution of least one adenine or guanine nucleotide in the sense or antisense region with an 8-bromoadenine or 8-bromoguanine nucleotide.

In some embodiments, the siRNA is conjugated to a non-nucleic acid molecule, wherein the non-nucleic acid molecule comprises a fatty acid, lipid, peptide, protein, antibody, or nanoparticle.

In some embodiments, the siRNA is conjugated to cholesterol.

In some embodiments, the oligonucleotide is capable of suppressing the expression of a Titin isoform N2BA to a lesser extent than the oligonucleotide is capable of suppressing the expression of a Titin isoform N2B.

In some embodiments, the suppression of the expression of the Titin isoform N2B is from about 2-fold to about 1000-fold greater than the suppression of the Titin isoform N2BA.

In some embodiments, the oligonucleotide, when administered to a cell, is capable of inhibiting the expression of the Titin isoform N2BA by 10% or less.

In some embodiments, the oligonucleotide is capable of increasing the ratio of Titin isoform N2BA to Titin isoform N2B in the cell.

In some embodiments, the ratio of the Titin isoform N2BA to the Titin isoform N2B is increased from about 1.2-fold to about 5-fold.

In some embodiments, the oligonucleotide is substantially complementary to a exon-exon boundary sequence formed by splicing of two non-sequential exons present in the TTN pre-mRNA transcript, wherein the exon-exon boundary sequence is present in the mature N2B mRNA transcript but not the mature N2BA mRNA transcript, and wherein the exon-exon boundary sequence is formed from splicing TTN exons 50 and 220.

In some embodiments, the oligonucleotide is substantially complementary to a exon-exon boundary sequence formed by splicing of two non-sequential exons present in the TTN pre-mRNA transcript, wherein the exon-exon boundary sequence is present in the mature N2B mRNA transcript but not the mature N2BA mRNA transcript, and wherein the exon-exon boundary sequence is formed from splicing TTN exons 10 and 12.

In some embodiments, the length of the exon-exon boundary sequence is from about 15 nucleotides to 50 nucleotides.

In some embodiments, the length of the exon-exon boundary sequence is from about 15 nucleotides to 30 nucleotides.

In some embodiments, the length of the exon-exon boundary sequence is from about 15 nucleotides to 27 nucleotides.

In some embodiments, the length of the exon-exon boundary sequence is from about 20 nucleotides to 27 nucleotides.

In another aspect, this disclosure features a pharmaceutical composition comprising any of the oligonucleotides described herein.

### 4. BRIEF DESCRIPTION OF THE DRAWINGS

Aspects, features, benefits, and advantages of the embodiments described herein will be apparent with regard to the following description, appended claims, and accompanying drawings. The drawings depict exemplary embodiments of the disclosure and are not intended to be limiting.

FIG. 1 shows a diagram of the mRNA of human Titin (TTN) (NM_003319.4) isoforms N2B spanning 3′ end of exon 50 and 5′ end of exon 220. Figure discloses SEQ ID NOS 51-53, respectively, in order of appearance.

FIG. 2 illustrates a strategy for targeting the target sequence of FIG. 1, targeting the overlapping TTN exon 50 and TTN exon 220 for the suppression of TTN N2B isoform mRNA.

FIGS. 3A-3E shows native gel electrophoresis for annealed siRNA complexes with different annealed duplexes corresponding to different siRNA targeting TTN isoform N2B in each lane. FIG. 3A shows the RNA ladder used in gels in FIGS. 3B-3D. FIGS. 3B-3D show the TBE 20% gradient gells loaded with 10 μmol of annealed duplexes per lane and stained with SybrGold dye with each lane containing annealed duplexes as noted in FIG. 3E. (RS)=re-synthesis.

FIG. 4 shows a nucleotide sequence used in a proxy reporter assay to identify oligonucleotides that target TTN isoform N2BA. Underlined sequence indicates 3′ end of Exon 50. Bold sequence indicates 5′ end of Exon 220.

FIG. 5 shows a nucleotide sequence used in a proxy reporter assay to identify oligonucleotides that target TTN isoform N2B. Underlined sequence indicates 3′ end of Exon 50. Bold sequence indicates 5′ end of Exon 220.

FIGS. 6A and 6B shows data generated in the proxy reporter assay (using the nucleotide sequence from FIG. 4) looking for oligonucleotides that target TTN isoform N2BA. FIG. 6A shows data as a histogram as relative normalized luciferase expression (percentage plasmid). Box with solid line indicates siRNA that failed to form a duplex (i.e., no siRNA was formed). RS refers to “re-synthesis” where duplex formation was achieved for TTN−6, TTN−4, and TTN-2. For RS data, TTN+0 (RS) and TTN+4 (RS) were also re-synthesized and transfected along with TTN−6, TTN−4, and TTN−2 even though duplex formation was achieved in the first synthesis for TTN+0 and TTN+4. FIG. 6B shows data from FIG. 6A in a table. “*” indicates siRNA that failed to form a duplex (i.e., no siRNA was formed).

FIGS. 7A-7B shows data generated in the proxy reporter assay (using the nucleotide sequence from FIG. 5) looking for oligonucleotides that target TTN isoform N2B. FIG. 7A shows data as a histogram as relative normalized luciferase expression (percentage plasmid). Box with solid line indicates siRNA that failed to form a duplex (i.e., no siRNA was formed). RS refers to “re-synthesis” where duplex formation was achieved for TTN−6, TTN−4, and TTN-2. For RS data, TTN+0 (RS) and TTN+4 (RS) were also re-synthesized and transfected along with TTN−6, TTN−4, and TTN−2 even though duplex formation was achieved in the first synthesis for TTN+0 and TTN+4. FIG. 7B shows data from FIG. 7A in a table. “*” indicates siRNA that failed to form a duplex (i.e., no siRNA was formed).

FIG. 8 shows a table of the ratio of Titin isoform N2BA to Titin isoform N2B from data in FIGS. 6A-6B and FIGS. 7A-7B. Ratios for TTN−6, TTN−4, and TTN−2 that did not form duplexes in the original synthesis (“*”) were not calculated.

### 5. DETAILED DESCRIPTION

The ability of the heart to function is partially dependent upon the ability of the cardiomyocyte (i.e., the contractile cell of the heart) to properly contract and relax. The ability of the cardiomyocyte to properly contract and relax is, in part, dependent upon the function of a specialized organelle called the sarcomere. The sarcomere is comprised of hundreds of proteins, of which one is called Titin. Titin, in humans, is encoded by the TTN gene. Sequences related to the TTN gene can be obtained from: cardiodb.org/titin/titin_transcripts.php. The contents of which are incorporated herein by reference and shown, in part, in Table 1.

TABLE 1
Titin Transcripts and Protein Isoforms

| | | Length | Number of | Refseq transcript |
|---|---|---|---|---|
| Transcript | Description | (aa) | exons | and protein |
| Meta | Inferred complete | 35991 | 363 | NM_001267550.1; |
| | meta-transcript | | | NP_001254479 |
| N2BA | Principle cardiac | 34350 | 313 | NM_001256850.1; |
| | long isoform | | | NP_001243779 |
| N2B | Principle cardiac | 26926 | 191 | NM_003319.4; |
| | short isoform | | | NP_003310.4 |
| N2A | Soleus/skeletal | 33423 | 312 | NM_133378.4; |
| | long isoform | | | NP_596869.4 |
| Novex-1 | Minor cardiac | 27051 | 192 | NM_133432.3; |
| | short isoform | | | NP_597676.3 |
| Novex-2 | Minor cardiac | 27118 | 192 | NM_133437.3; |
| | short isoform | | | NP_597681.3 |
| Novex-3 | Minor small | 5604 | 46 | NM_133379.3; |
| | cardiac isoform | | | NP_596870.2 |

A number of Titin isoforms are generated by alternative splicing of the TTN pre-mRNA. The major cardiac Titin isoforms are Titin isoform N2BA and Titin isoform N2B. The isoform designations (N2BA and N2B) are based on protein motifs called N2BA and N2B. Titin isoform N2BA has both N2BA and N2B motifs, whereas Titin isoform N2B has only the N2B motif. Titin can be thought of as a molecular spring. With regards to the two major Titin isoforms, Titin isoform N2BA is considered to be “springy” (i.e., more compliant in comparison to Titin isoform N2B), whereas Titin isoform N2B is considered to be less “springy” (i.e., less-compliant in comparison to Titin isoform N2BA) (See, e.g., Freiburg A, et al., Circ Res. 2000 Jun. 9; 86(11):1114-21).

Titin isoform N2BA and Titin isoform N2B exist in a ratio in the sarcomere. In adult humans, Titin isoform N2BA is the primary isoform with Titin N2B being less abundant. A change in the Titin isoform ratio positively correlates with both left ventricle cardiac systolic and diastolic dysfunction. For instance, in humans with systolic dysfunction (i.e., a heart that cannot contract properly, as seen in dilated cardiomyopathy), the ratio of Titin isoform N2BA to Titin isoform N2B may be increased relative to humans without systolic dysfunction. In contrast, in humans with, and in animal models of, diastolic dysfunction (e.g., a heart that cannot relax properly) the ratio of Titin isoform N2BA to Titin isoform N2B is decreased relative to humans and animal models without diastolic dysfunction.

Because modulating Titin isoform expression may be therapeutically beneficial to treat heart failure and other diseases, the present compositions and methods have been developed to modulate the expression and relative expression of Titin protein isoforms such as, for example, N2B and/or N2BA.

Accordingly, the present disclosure provides compositions and methods for reducing the expression or inhibiting Titin isoform N2B, optionally without reducing the expression or inhibiting Titin isoform N2BA or reducing the expression or inhibiting Titin isoform N2BA by a lesser extent than for Titin isoform N2B. This represents a novel strategy to improve diastolic function in subjects with diastolic heart failure (DHF). The disclosure further provides methods and compositions for increasing the ratio of Titin isoform N2BA protein to Titin isoform N2B protein. Broadly, the present disclosure provides oligonucleotides which can differentially bind to mRNA of one Titin isoform, modulate translation thereof, and thereby alter the relative amounts of the isoforms produced in a cell that has been treated with the oligonucleotides.

In certain embodiments, the composition containing the oligonucleotide(s) can be delivered to a subject, preferably a mammal. Non-limiting examples of a mammal treatable according to the methods of the current disclosure include mouse, rat, dog, guinea pig, cow, horse, donkey, mule, cat, rabbit, pig, monkey, ape, chimpanzee, baboon, and particularly a human. Mammals treatable with the methods of the current disclosure include those mammals comprising heart muscle and cardiomyocytes expressing Titin isoforms encoded by mRNAs comprising sequences that could be targeted by isoform-specific oligonucleotides as described herein.

### 5.1 Definitions

As used herein, the term “diastolic dysfunction” refers to the inability for the heart to properly relax during diastole which is the segment of the cardiac pumping cycle in which the left heart ventricle fills with oxygenate blood returning from the lungs.

As used herein, the term “diastolic heart failure” (or “DHF”) refers to the clinical symptoms of heart failure that are brought on upon diastolic dysfunction”.

As used herein, the term “exon-exon boundary sequence” refers to a sequence resulting from a splicing even of two exons that produces a new, continuous, nucleotide sequence which is not present in the pre-mRNA. For instance, TTN exons 49 and 50 are spliced together during the generation of Titin isoform N2BA mRNA to form an exon-exon boundary sequence. In another instance, TTN exons 50 and 220 are spliced together during the generation of Titin isoform N2B mRNA but are not spliced together during the generation of Titin isoform N2BA mRNA. Thus, the exon-exon boundary sequence of exon 50 and exon 220 is present in Titin isoform N2B mRNA but not in Titin isoform N2BA mRNA.

As used herein, the terms “treatment, treating, treat” or equivalents of these terms refer to healing, alleviating, relieving, altering, remedying, ameliorating, improving, or affecting the condition or the symptoms of a subject suffering with a disease, for example, a cardiac disorder. The subject to be treated can be suffering from or at risk of developing the disorder, for example, a cardiac disorder, including, for example, congestive heart failure or be at risk of developing congestive heart failure.

As used herein, the terms “preventing, preventive, prophylactic” or equivalents of these terms indicate that the oligonucleotide is provided in advance of any disease symptoms and are a separate aspect of the present disclosure (i.e., an aspect of the present disclosure that is distinct from aspects related to the terms “treatment, treating, treat” or equivalents of these terms which refer to healing, alleviating, relieving, altering, remedying, ameliorating, improving, or affecting the condition or the symptoms of a subject suffering with heart disease, for example, a chronic heart failure). The prophylactic administration of the oligonucleotide serves to prevent or attenuate any subsequent symptoms or disease.

By “therapeutically effective dose,” “therapeutically effective amount”, or “effective amount” is intended to be an amount of an oligonucleotide or pharmaceutical composition comprising the oligonucleotide disclosed herein that, when administered to a subject, decreases the incident of heart failure, or improves diastolic function as compared to untreated subjects. “Positive therapeutic response” refers to, for example, improving the condition of at least one of the symptoms of a heart disorder.

The term “unit dose” refers to a physically discrete unit suitable for use in a subject, each unit containing a predetermined quantity of the therapeutic composition calculated to produce the desired response in association with its administration, i.e., the appropriate route and treatment regimen. In specific embodiments, it may be desirable to administer the oligonucleotide of the present disclosure in the range of about 0.01 mg/kg to about 20 mg/kg such as, for example, about 0.05 mg/kg, about 0.1 mg/kg, about 0.5 mg·kg, about 1 mg/kg, about 2 mg/kg, about 3 mg/kg, about 4 mg·kg, about 5 mg/kg, about 10 mg/kg, or about 15 mg/kg.

In some embodiments of the present disclosure, the method comprises administration of multiple doses of the oligonucleotides. The method may comprise administration of 1, 2, 3, 4, 5, 6, 7, 8, 9, 10, 15, 20, 25, 30, 35, 40, or more therapeutically effective doses of a composition comprising the oligonucleotide as described herein. In some embodiments, doses are administered over the course of 1 day, 2 days, 3 days, 4 days, 5 days, 6 days, 7 days, 10 days, 14 days, 21 days, 30 days, or more than 30 days. The frequency and duration of administration of multiple doses of the compositions is such as to improve cardiac function and thereby treat or prevent the incidence of heart failure. Moreover, treatment of a subject with a therapeutically effective amount of the oligonucleotide of the present disclosure can include a single treatment or can include a series of treatments. Treatment may proceed for the lifetime of the patient. It will also be appreciated that the effective dosage of an oligonucleotide can be used for treatment may increase or decrease over the course of a particular treatment. Changes in dosage may result and become apparent from the results of diagnostic assays for detecting heart failure and/or diastolic function.

As used herein, the term “nucleic acid,” “oligonucleotide,” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

As used herein, the term “isolated nucleic acid” molecule refers to a nucleic acid molecule that is separated from other nucleic acid molecules that are usually associated with the isolated nucleic acid molecule. Thus, an “isolated nucleic acid molecule” includes, without limitation, a nucleic acid molecule that is free of nucleotide sequences that naturally flank one or both ends of the nucleic acid in the genome of the organism from which the isolated nucleic acid is derived (e.g., a cDNA or genomic DNA fragment produced by PCR or restriction endonuclease digestion). Such an isolated nucleic acid molecule is generally introduced into a vector (e.g., a cloning vector or an expression vector) for convenience of manipulation or to generate a fusion nucleic acid molecule. In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA or genomic library) or a gel (e.g., agarose, or polyacrylamide) containing restriction-digested genomic DNA, is not an “isolated nucleic acid”.

As used herein, the term “gene” means a segment of DNA that encodes a polypeptide chain or a non-coding RNA; “gene” also includes regions preceding and following the coding region (e.g., the promoter, introns, and 5′ and 3′ untranslated regions) that can be involved in the transcription/translation of the gene product and the regulation of the transcription/translation.

As used herein, the terms “polypeptide”, “peptide”, and “protein” are used interchangeably herein to refer to a polymer of amino acids. The terms apply to amino acid polymers in which one or more amino acid residues are artificial chemical mimetic of a corresponding naturally occurring amino acids, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used herein, the terms “identical” or percent “identity”, in the context of describing two or more polynucleotide or amino acid sequences, refer to two or more sequences or subsequences that are the same or have a specified percentage of amino acid residues or nucleotides that are the same (for example, an oligonucleotide used in the method of this present disclosure has at least 80% sequence identity, preferably 85%, 90%, 91%, 92%, 93, 94%, 95%, 96%, 97%, 98%, 99%, or 100% identity, to a reference sequence), when compared and aligned for maximum correspondence over a comparison window, or designated region as measured using one of the following sequence comparison algorithms or by manual alignment and visual inspection. Such sequences are then said to be “substantially identical”. With regard to polynucleotide sequences, this definition also refers to the complement of a sequence. The comparison window, in certain embodiments, refers to the full length sequence of a given mRNA sequence or polypeptide.

As used herein, the singular forms “a”, “an” and “the” are intended to include the plural forms as well, unless the context clearly indicates otherwise. Furthermore, to the extent that the terms “contain,” “including”, “includes”, “having”, “has”, “with”, or variants thereof are used in either the detailed description and/or the claims, such terms are intended to be inclusive in a manner similar to the term “comprising”. For example, “a composition comprising an oligonucleotide” may or may not contain additional, unrecited components.

The phrases “consisting essentially of” or “consists essentially of” indicate that the subject being described encompasses embodiments containing only the specified materials or steps and also embodiments that contain the specified materials or steps and additional materials or steps that do not materially affect the any of the performance, functioning, or properties of the subject.

The phrases “consisting of” or “consists of” indicate that the subject being described encompasses embodiments containing only the specified materials or steps.

The term “about” means within an acceptable error range for the particular value as determined by one of ordinary skill in the art. In the context of compositions containing amounts of ingredients where the terms “about” is used, these compositions contain the stated amount of the ingredient with a variation (error range) of 0-10% around the value (X±10%). In other contexts the term “about” is provides a variation (error range) of 0-10% around a given value (X±10%). As is apparent, this variation represents a range that is up to 10% above or below a given value.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

An endogenous nucleic acid is a nucleic acid that is naturally present in a cell. For example, a nucleic acid present in the genomic DNA of a cell is an endogenous nucleic acid.

An exogenous nucleic acid is any nucleic acid that is not naturally present in a cell. For example, a nucleic acid vector introduced into a cell constitutes an exogenous nucleic acid. Other examples of an exogenous nucleic acid include the vectors comprising a heterologous promoter linked to an endogenous nucleic acid, e.g., a nucleic acid encoding a kinase.

In certain embodiments, oligonucleotides of the present disclosure comprise oligonucleotides that are designed to “suppress,” “reduce,” (or the like) the expression of a gene (or isoform of a gene) at the mRNA and/or protein level. For example TTN isoform N2B expression may be “reduced” or “suppressed” by an oligonucleotide described herein. This refers to a detectable decrease of gene expression, as compared to a non-treated subjects (e.g., experimental control cells, tissues, organs, or organisms). Thus, subjects that are treated with the nucleotide-containing compositions can exhibit reduced or suppressed expression of a gene, exhibit a reduction in gene expression or expression of active protein that can range from about 30% to about 99.99%, or are devoid of expression (expression is abolished) of the gene or an active protein encoded by the gene.

As used herein, a “pharmaceutical” refers to a compound manufactured for use as a medicinal and/or therapeutic drug in human or animal patients or subjects.

### 5.2 Oligonucleotide Compositions

The present disclosure provides oligonucleotides, including a small interfering RNA (siRNA), an aptamer, a short hairpin RNA (shRNA), or a micro RNA (miRNA) which is capable of specifically binding at least some of the Titin isoform mRNAs. In some embodiments, the oligonucleotide may be a single-stranded or a double-stranded nucleic acid molecule.

In some embodiments, the siRNA is a self-delivering RNA (sdRNA). In some embodiments, a self-delivering is as described in U.S. Pat. Nos. 8,794,443; 9,175,289; 10,774,330; PCT Publication Nos: WO 2010/033247 and WO 2021/138537, each of which is herein incorporated by reference in its entirety. In some embodiments, a sdRNA is an isolated asymmetric double stranded nucleic acid molecule comprising a guide strand (e.g., an antisense strand) with a minimal length of 16 nucleotides, and a passenger strand (e.g., sense strand) of 8-18 nucleotides in length, wherein the double stranded nucleic acid molecule has a double stranded region and a single stranded region, the single stranded region having 4-12 nucleotides in length and having one or more nucleotide backbone modifications.

Any TTN isoform or group of isoforms may be targeted for reduced expression by an oligonucleotide of the present disclosure, as long as an appropriate specific target sequence can be found in the targeted isoforms (e.g., an exon-exon boundary sequence, discussed below). Thus, therapeutic treatments can be accomplished via modulating relative amounts of the various isoforms. In some embodiments, the target of the oligonucleotides is TTN isoform N2B, and the relative amount of N2B isoform protein/mRNA is reduced. In some embodiments, the target of the oligonucleotides is TTN isoform N2BA, and the relative amount of N2BA isoform protein/mRNA is reduced. As noted, Titin isoform N2BA and Titin isoform N2B are generated from alternative splicing of the same TTN pre-mRNA transcript. All exons present in the mature Titin isoform N2B mRNA are also present in the mature Titin isoform N2BA mRNA. Thus, targeting nucleotide sequences internal to a TTN exon required for generation of Titin isoform N2B mRNA will also result in the inhibition of the Titin isoform N2BA mRNA. Accordingly, this strategy is not predicted to result in an increase in the Titin isoform N2BA to Titin isoform N2B ratio. It is noted that whenever exon numbers are used herein, the numbers refer to the Locus Reference Genomic (LRG) numbering for the TTN transcript (e.g., all exons numbered in the order they occur on the genome, from 5′ to 3′), unless explicitly noted otherwise.

In some cases, splicing of 2 exons can produce a new, continuous, nucleotide sequence that is not present in the pre-mRNA, which is referred to as the “exon-exon boundary sequence”. For instance, TTN exons 49 and 50 are spliced together during the generation of Titin isoform N2BA mRNA. Yet, TTN exons 49 and 50 are also spliced together during the generation of Titin isoform N2B. Thus, targeting an exon-exon boundary sequence, with, for example, a siRNA, for the inhibition of Titin isoform N2B can also result in the inhibition of Titin isoform N2BA. In contrast, TTN exons 50 and 220 are spliced together during the generation of Titin isoform N2B mRNA but are not spliced together during the generation of Titin isoform N2BA mRNA. Thus, the exon-exon boundary sequence of exon 50 and exon 220 is present in Titin isoform N2B mRNA but not in Titin isoform N2BA mRNA. As a result, the exon-exon boundary sequence spanning exon 50 and exon 220 (and fragments thereof such as SEQ ID NO: 31 (UGUACAGCCACACUAACUGUGACAGUGCCUGGAGGUGAAAAGAAAGU) and SEQ ID NO: 32 (UGACAGUGCCUGGAGGUGAA) can be a potential target for a siRNA, to differentially inhibit Titin isoform N2B while having little or no inhibitory effect of Titin isoform N2BA expression.

Thus, it is understood that isoform N2B may be targeted with specificity by targeting any sequences present in the N2B isoform (or present in the Novex-1 or Novex-2 isoforms), but not present in other TTN isoforms (e.g., N2BA).

In some embodiments, the exon-exon boundary sequences are about 15 to about 50, about 15 to about 30, about 15 to about 27, about 17 to about 22, about 25 to about 30 or about 20 to about 27 nucleotides in length. In some embodiments, the oligonucleotides that bind the exon-exon boundary sequences are about 15 to about 50, about 15 to about 30, about 15 to about 27, about 17 to about 22, about 25 to about 30 or about 20 to about 27 nucleotides in length.

In some embodiments, the oligonucleotides can target (e.g., be complementary to) an exon-exon boundary sequence of a TTN isoform comprising at least one of SEQ ID NOs: 31 or 32 (including both SEQ ID NOs: 31 and 32), or the nucleotide having at least 75%, 80%, 85%, 90%, 91%, 92%, 93%, 94%, 95%, 96%, 97%, 98%, or 99%, or 100% sequence identity to the sequence of SEQ ID NOs: 31 or 32.

In some embodiments, the oligonculdoetide can target (e.g., be complementary to) an exon-exon boundary sequence of TTN isoform novex-1 or novex-2.

In some embodiments, the oligonucleotides targeting the TTN isoforms according to the current disclosure comprise at least 75% sequence complementarity to at least one sequence of SEQ ID NOs: 31 or 32. In another embodiment, the oligonucleotides targeting the TTN isoforms according to the current disclosure comprise at least 80% sequence complementarity to at least one sequence of SEQ ID NOs: 31 or 32. In another embodiment, the oligonucleotides targeting the TTN isoforms according to the current disclosure comprise at least 85% sequence complementarity to at least one sequence of SEQ ID NOs: 31 or 32. In another embodiment, the oligonucleotides targeting the TTN isoforms according to the current disclosure comprise at least 90% sequence complementarity to at least one sequence of SEQ ID NOs: 31 or 32. In another embodiment, the oligonucleotides targeting the TTN isoforms according to the current disclosure comprise at least 91% sequence complementarity to at least one sequence of SEQ ID NOs: 31 or 32. In another embodiment, the oligonucleotides targeting the TTN isoforms according to the current disclosure comprise at least 92% sequence complementarity to at least one sequence of SEQ ID NOs: 31 or 32. In another embodiment, the oligonucleotides targeting the TTN isoforms according to the current disclosure comprise at least 93% sequence complementarity to at least one sequence of SEQ ID NOs: 31 or 32. In another embodiment, the oligonucleotides targeting the TTN isoforms according to the current disclosure comprise at least 94% sequence complementarity to at least one sequence of SEQ ID NOs: 31 or 32. In another embodiment, the oligonucleotides targeting the TTN isoforms according to the current disclosure comprise at least 95% sequence complementarity to at least one sequence of SEQ ID NOs: 31 or 32. In another embodiment, the oligonucleotides targeting the TTN isoforms according to the current disclosure comprise at least 96% sequence complementarity to at least one sequence of SEQ ID NOs: 31 or 32. In another embodiment, the oligonucleotides targeting the TTN isoforms according to the current disclosure comprise at least 97% sequence complementarity to at least one sequence of SEQ ID NOs: 31 or 32. In another embodiment, the oligonucleotides targeting the TTN isoforms according to the current disclosure comprise at least 98% sequence complementarity to at least one sequence of SEQ ID NOs: 31 or 32. In another embodiment, the oligonucleotides targeting the TTN isoforms according to the current disclosure comprise at least 99% sequence complementarity to at least one sequence of SEQ ID NOs: 31 or 32. In another embodiment, the oligonucleotides targeting the TTN isoforms according to the current disclosure comprise at least 100% sequence complementarity to at least one sequence of SEQ ID NOs: 31 or 32.

The oligonucleotides targeting the TTN isoforms can have sufficient sequence complementarity to the targeted exon-exon boundary sequences, including SEQ ID NOs: 31 or 32, to mediate target-specific inhibition. For example, the oligonucleotides can hybridize with the target mRNA. The hybridization can lead to steric hindrance of translation of the mRNA, modification of the mRNA, sequestration of the mRNA, nucleolytic degradation of the mRNA, or any combination thereof. Thus, targeted mRNAs may produce less protein, leading to alteration of the relative amounts of titin isoforms in a cell treated with the oligonucleotides.

### 5.2.1 Oligonucleotides Targeting Titin Isoform N2B

In some embodiments, the oligonucleotide can comprise a siRNA that targets TTN isoform N2B with little to no effect on TTN isoform N2BA. In some embodiments, the siRNA can comprise a circular siRNA construct. In some embodiments, the siRNA is a self-deliverable SiRNA (sdRNA).

In some embodiments, the methods described herein utilize the exon 50-exon 220 boundary sequence present in Titin isoform N2B but not present in Titin isoform N2BA, such as, for example SEQ ID NOs: 31 or 32, to achieve inhibition of Titin isoform N2B via a hybridizable oligonucleotide (e.g., siRNA), whereby inhibition increases the ratio of Titin isoform N2BA to Titin isoform N2B, and improves diastolic function in patients with diastolic heart failure (DHF).

In some embodiments, the siRNA (e.g., the sdRNA) is capable of reducing TTN isoform N2B transcript expression in the patient, cardiomyocyte, or cell by at least about 20% (e.g., by at least about 30%, by least about 40%, by at least about 50%, by at least about 60%, by at least about 70%, by at least about 80%, or by at least 90%) compared to a corresponding patient, cardiomyocyte, or cell not exposed to the siRNA.

In some embodiments, the siRNA (e.g., the sdRNA) is double stranded. In some embodiments, the siRNA comprises a sense strand and an antisense strand. In some embodiments, each strand of the siRNA includes a sequence between about 10 to about 40 nucleotides (e.g., about 10 to about 35, about 10 to about 30, about 10 to about 20, about 10 to about 15, about 15 to about 40, about 15 to about 35, about 15 to about 30, about 15 to about 25, about 15 to about 20, about 20 to about 40, about 20 to about 35, about 20 to about 30, about 30 to about 25, about 25 to about 40, about 25 to about 35, about 25 to about 30, about 30 to about 40, about 30 to about 35, or about 35 to about 50 nucleotides) in length. In some embodiments, the sense strand and the antisense strand are equal lengths. In some embodiments, the sense strand and the antisense strand are unequal lengths.

In some embodiments, the antisense strand includes a sequence between about 15 to about 40 (or any of the subranges included therein) nucleotides in length.

In some embodiments, the dsRNA molecule (e.g., the siRNA) comprises a sense strand and an antisense strand, each strand having 10 to 40 nucleotides, wherein the antisense strand has sufficient complementarity to the target sequence to mediate RNA interference.

In some embodiments, the siRNA comprises a polynucleotide sequence having at least about 80%, at least about 85%, at least about 90%, at least about 95%, or about 100% complementarity to the nucleic acid sequence within the exon-exon boundary sequence of SEQ ID NO: 31.

In some embodiments, the siRNA (e.g., the sdRNA) includes an antisense strand having at least 80%, at least 85%, at least 90%, at least 95%, or at least 99% sequence identity to a sequence selected from SEQ ID NO: 2, 4, 6, 8, 10, 12, 14, 22, 24, 26, 28, and 30. In some embodiments, the the antisense region has a sequence selected from SEQ ID NOs: 20, 22, 24, 26, 28, and 30. In some embodiments, the antisense region has a sequence selected from SEQ ID NOs: 22, 24, and 26. In some embodiments, the antisense region is SEQ ID NO: 22. In some embodiments, the antisense region is SEQ ID NO: 26. In particular embodiments, the antisense region is SEQ ID NO: 24.

In some embodiments, the siRNA (e.g., sdRNA) includes a sense strand having at least 80%, at least 85%, at least 90%, at least 95%, or at least 99% sequence identity to a sequence selected from SEQ ID NO: 1, 3, 5, 7, 9, 11, 13, 21, 23, 25, 27, or 29. See Table 2 for siRNA sequences.

TABLE 2
SiRNA Targeting TTN Isoform N2B

| SEQ | | | 3′ |
|---|---|---|---|
| ID NO: | Identifier | Sequence | modification |
| 1 | TTN-7 (sense) (5′-3′) | UAACUGUGACAGUGA | TEG-Chol |
| 2 | TTN-7 (antisense) (3′-5′) | GUGUGAUUGACACUGUCACU | — |
| 3 | TTN-6 (sensc) (5′-3′) | AACUGUGACAGUGCA | TEG-Chol |
| 4 | TTN-6 (antisense) (3′-5′) | UGUGAUUGACACUGUCACGU | — |
| 5 | TTN-5 (sense) (5′-3′) | ACUGUGACAGUGCCA | TEG-Chol |
| 6 | TTN-5 (antisense) (3′-5′) | GUGAUUGACACUGUCACGGU | — |
| 7 | TTN-4 (sense) (5′-3′) | CUGUGACAGUGCCUA | TEG-Chol |
| 8 | TTN-4 (antisense) (3′-5′) | UGAUUGACACUGUCACGGAU | — |
| 9 | TTN-3 (sense) (5′-3′) | UGUGACAGUGCCUGA | TEG-Chol |
| 10 | TTN-3 (antisense) (3′-5′) | GAUUGACACUGUCACGGACU | — |
| 11 | TTN-2 (sense) (5′-3′) | GUGACAGUGCCUGGA | TEG-Chol |
| 12 | TTN-2 (antisense) (3′-5′) | AUUGACACUGUCACGGACCU | — |
| 13 | TTN-1 (sense) (5′-3′) | UGACAGUGCCUGGAA | TEG-Chol |
| 14 | TTN-1 (antisense) (3′-5′) | UUGACACUGUCACGGACCUU | — |
| 15 | TTN-0 (sense) (5′-3′) | GACAGUGCCUGGAGA | TEG-Chol |
| 16 | TTN-0 (antisense) (3′-5′) | UGACACUGUCACGGACCUCU | — |
| 17 | TTN+1 (sense) (5′-3′) | ACAGUGCCUGGAGGA | TEG-Chol |
| 18 | TTN+1 (antisensc) (3′-5′) | GACACUGUCACGGACCUCCU | — |
| 19 | TTN+2 (sense) (5′-3′) | CAGUGCCUGGAGGUA | TEG-Chol |
| 20 | TTN+2 (antisense) (3′-5′) | ACACUGUCACGGACCUCCAU | — |
| 21 | TTN+3 (sense) (5′-3′) | AGUGCCUGGAGGUGA | TEG-Chol |
| 22 | TTN+3 (antisense) (3′-5′) | CACUGUCACGGACCUCCACU | — |
| 23 | TTN+4 (sense) (5′-3′) | GUGCCUGGAGGUGAA | TEG-Chol |
| 24 | TTN+4 (antisense) (3′-5′) | ACUGUCACGGACCUCCACUU | — |
| 25 | TTN+5 (sensc) (5′-3′) | UGCCUGGAGGUGAAA | TEG-Chol |
| 26 | TTN+5 (antisense) (3′-5′) | CUGUCACGGACCUCCACUUU | — |
| 27 | TTN+6 (sense) (5′-3′) | GCCUGGAGGGAAAA | TEG-Chol |
| 28 | TTN+6 (antisense) (3′-5′) | UGUCACGGACCUCCACUUUU | — |
| 29 | TTN+7 (sense) (5′-3′) | CCUGGAGGUGAAAAA | TEG-Chol |
| 30 | TTN+7 (antisense) (3′-5′) | GUCACGGACCUCCACUUUUU | — |

In some embodiments, the siRNA (e.g., the sdRNA) directed against TTN isoform N2B (and/or Novex-1 and/or Novex-2) includes an antisense strand having at least 85%, at least 90%, at least 95%, at least 99% sequence identity to SEQ ID NO: 2; and a sense strand having at least 85%, at least 90%, at least 95%, at least 99% sequence identity to SEQ ID NO: 1, wherein the antisense strand hybridizes with the sense strand. In some embodiments, the siRNA (e.g., the sdRNA) directed against TTN isoform N2B (and/or Novex-1 and/or Novex-2) includes an antisense strand of SEQ ID NO: 2; and a sense strand of SEQ ID NO: 1, wherein the antisense strand is hybridized with the sense strand.

In some embodiments, the siRNA (e.g., the sdRNA) directed against TTN isoform N2B (and/or Novex-1 and/or Novex-2) includes an antisense strand having at least 85%, at least 90%, at least 95%, at least 99% sequence identity to SEQ ID NO: 4; and a sense strand having at least 85%, at least 90%, at least 95%, at least 99% sequence identity to SEQ ID NO: 3, wherein the antisense strand hybridizes with the sense strand. In some embodiments, the siRNA (e.g., the sdRNA) directed against TTN isoform N2B (and/or Novex-1 and/or Novex-2) includes an antisense strand of SEQ ID NO: 4; and a sense strand of SEQ ID NO: 3, wherein the antisense strand is hybridized with the sense strand.

In some embodiments, the siRNA (e.g., the sdRNA) directed against TTN isoform N2B (and/or Novex-1 and/or Novex-2) includes an antisense strand having at least 85%, at least 90%, at least 95%, at least 99% sequence identity to SEQ ID NO:6; and a sense strand having at least 85%, at least 90%, at least 95%, at least 99% sequence identity to SEQ ID NO: 5, wherein the antisense strand hybridizes with the sense strand. In some embodiments, the siRNA (e.g., the sdRNA) directed against TTN isoform N2B (and/or Novex-1 and/or Novex-2) includes an antisense strand of SEQ ID NO: 6; and a sense strand of SEQ ID NO: 5, wherein the antisense strand is hybridized with the sense strand.

In some embodiments, the siRNA (e.g., the sdRNA) directed against TTN isoform N2B (and/or Novex-1 and/or Novex-2) includes an antisense strand having at least 85%, at least 90%, at least 95%, at least 99% sequence identity to SEQ ID NO: 8; and a sense strand having at least 85%, at least 90%, at least 95%, at least 99% sequence identity to SEQ ID NO: 7, wherein the antisense strand hybridizes with the sense strand. In some embodiments, the siRNA (e.g., the sdRNA) directed against TTN isoform N2B (and/or Novex-1 and/or Novex-2) includes an antisense strand of SEQ ID NO: 8; and a sense strand of SEQ ID NO: 7, wherein the antisense strand is hybridized with the sense strand.

In some embodiments, the siRNA (e.g., the sdRNA) directed against TTN isoform N2B (and/or Novex-1 and/or Novex-2) includes an antisense strand having at least 85%, at least 90%, at least 95%, at least 99% sequence identity to SEQ ID NO: 10; and a sense strand having at least 85%, at least 90%, at least 95%, at least 99% sequence identity to SEQ ID NO: 9, wherein the antisense strand hybridizes with the sense strand. In some embodiments, the siRNA (e.g., the sdRNA) directed against TTN isoform N2B (and/or Novex-1 and/or Novex-2) includes an antisense strand of SEQ ID NO: 10; and a sense strand of SEQ ID NO: 9, wherein the antisense strand is hybridized with the sense strand.

In some embodiments, the siRNA (e.g., the sdRNA) directed against TTN isoform N2B (and/or Novex-1 and/or Novex-2) includes an antisense strand having at least 85%, at least 90%, at least 95%, at least 99% sequence identity to SEQ ID NO: 12; and a sense strand having at least 85%, at least 90%, at least 95%, at least 99% sequence identity to SEQ ID NO: 11, wherein the antisense strand hybridizes with the sense strand. In some embodiments, the siRNA (e.g., the sdRNA) directed against TTN isoform N2B (and/or Novex-1 and/or Novex-2) includes an antisense strand of SEQ ID NO: 12; and a sense strand of SEQ ID NO: 11, wherein the antisense strand is hybridized with the sense strand.

In some embodiments, the siRNA (e.g., the sdRNA) directed against TTN isoform N2B (and/or Novex-1 and/or Novex-2) includes an antisense strand having at least 85%, at least 90%, at least 95%, at least 99% sequence identity to SEQ ID NO: 14; and a sense strand having at least 85%, at least 90%, at least 95%, at least 99% sequence identity to SEQ ID NO: 13, wherein the antisense strand hybridizes with the sense strand. In some embodiments, the siRNA (e.g., the sdRNA) directed against TTN isoform N2B (and/or Novex-1 and/or Novex-2) includes an antisense strand of SEQ ID NO: 14; and a sense strand of SEQ ID NO: 13, wherein the antisense strand is hybridized with the sense strand.

In some embodiments, the siRNA (e.g., the sdRNA) directed against TTN isoform N2B (and/or Novex-1 and/or Novex-2) includes an antisense strand having at least 85%, at least 90%, at least 95%, at least 99% sequence identity to SEQ ID NO: 16; and a sense strand having at least 85%, at least 90%, at least 95%, at least 99% sequence identity to SEQ ID NO: 15, wherein the antisense strand hybridizes with the sense strand. In some embodiments, the siRNA (e.g., the sdRNA) directed against TTN isoform N2B (and/or Novex-1 and/or Novex-2) includes an antisense strand of SEQ ID NO: 16; and a sense strand of SEQ ID NO: 15, wherein the antisense strand is hybridized with the sense strand.

In some embodiments, the siRNA (e.g., the sdRNA) directed against TTN isoform N2B (and/or Novex-1 and/or Novex-2) includes an antisense strand having at least 85%, at least 90%, at least 95%, at least 99% sequence identity to SEQ ID NO: 20; and a sense strand having at least 85%, at least 90%, at least 95%, at least 99% sequence identity to SEQ ID NO: 19, wherein the antisense strand hybridizes with the sense strand. In some embodiments, the siRNA (e.g., the sdRNA) directed against TTN isoform N2B (and/or Novex-1 and/or Novex-2) includes an antisense strand of SEQ ID NO: 20; and a sense strand of SEQ ID NO: 19, wherein the antisense strand is hybridized with the sense strand.

In some embodiments, the siRNA (e.g., the sdRNA) directed against TTN isoform N2B (and/or Novex-1 and/or Novex-2) includes an antisense strand having at least 85%, at least 90%, at least 95%, at least 99% sequence identity to SEQ ID NO: 22; and a sense strand having at least 85%, at least 90%, at least 95%, at least 99% sequence identity to SEQ ID NO: 21, wherein the antisense strand hybridizes with the sense strand. In some embodiments, the siRNA (e.g., the sdRNA) directed against TTN isoform N2B (and/or Novex-1 and/or Novex-2) includes an antisense strand of SEQ ID NO: 22; and a sense strand of SEQ ID NO: 21, wherein the antisense strand is hybridized with the sense strand.

In some embodiments, the siRNA (e.g., the sdRNA) directed against TTN isoform N2B (and/or Novex-1 and/or Novex-2) includes an antisense strand having at least 85%, at least 90%, at least 95%, at least 99% sequence identity to SEQ ID NO: 24; and a sense strand having at least 85%, at least 90%, at least 95%, at least 99% sequence identity to SEQ ID NO: 23, wherein the antisense strand hybridizes with the sense strand. In some embodiments, the siRNA (e.g., the sdRNA) directed against TTN isoform N2B (and/or Novex-1 and/or Novex-2) includes an antisense strand of SEQ ID NO: 24; and a sense strand of SEQ ID NO: 23, wherein the antisense strand is hybridized with the sense strand.

In some embodiments, the siRNA (e.g., the sdRNA) directed against TTN isoform N2B (and/or Novex-1 and/or Novex-2) includes an antisense strand having at least 85%, at least 90%, at least 95%, at least 99% sequence identity to SEQ ID NO: 26; and a sense strand having at least 85%, at least 90%, at least 95%, at least 99% sequence identity to SEQ ID NO: 25, wherein the antisense strand hybridizes with the sense strand. In some embodiments, the siRNA (e.g., the sdRNA) directed against TTN isoform N2B (and/or Novex-1 and/or Novex-2) includes an antisense strand of SEQ ID NO: 26; and a sense strand of SEQ ID NO: 25, wherein the antisense strand is hybridized with the sense strand.

In some embodiments, the siRNA (e.g., the sdRNA) directed against TTN isoform N2B (and/or Novex-1 and/or Novex-2) includes an antisense strand having at least 85%, at least 90%, at least 95%, at least 99% sequence identity to SEQ ID NO: 28; and a sense strand having at least 85%, at least 90%, at least 95%, at least 99% sequence identity to SEQ ID NO: 27, wherein the antisense strand hybridizes with the sense strand. In some embodiments, the siRNA (e.g., the sdRNA) directed against TTN isoform N2B (and/or Novex-1 and/or Novex-2) includes an antisense strand of SEQ ID NO: 28; and a sense strand of SEQ ID NO: 27, wherein the antisense strand is hybridized with the sense strand.

In some embodiments, the siRNA (e.g., the sdRNA) directed against TTN isoform N2B (and/or Novex-1 and/or Novex-2) includes an antisense strand having at least 85%, at least 90%, at least 95%, at least 99% sequence identity to SEQ ID NO: 30; and a sense strand having at least 85%, at least 90%, at least 95%, at least 99% sequence identity to SEQ ID NO: 29, wherein the antisense strand hybridizes with the sense strand. In some embodiments, the siRNA (e.g., the sdRNA) directed against TTN isoform N2B (and/or Novex-1 and/or Novex-2) includes an antisense strand of SEQ ID NO: 30; and a sense strand of SEQ ID NO: 29, wherein the antisense strand is hybridized with the sense strand.

### 5.2.2 siRNA Modifications

In some instances, the oligonucleotides (e.g., siRNAs) can be degraded in vivo as a result of its cleavage by endonucleases on pyrimidines and exonucleases from both the 3′ and 5′ ends. Therefore, the oligonucleotide can include modifications including modified nucleotides that increase resistance to endonuclease and exonuclease-mediated degradation.

In some embodiments, the oligonucleotide molecule (e.g., the siRNA targeting Titin isoform N2B) comprises at least one (e.g., at least 2, at least 3, at least 4, at least 5, at least 6, at least 7, at least 8, at least 9, or ten or more) modified nucleotide.

In some embodiments, a modified nucleotide includes a modification at the 2′-position, the phosphate linkage, the ribose, the nucleobase, or any combination thereof.

Modified nucleotides may be selected from nucleobase-, sugar- and backbone-modified residues and combinations thereof. In certain embodiments, sugar-modified building blocks, particularly sugar-modified ribonucleotide building blocks can be used in the oligonucleotides that target TTN mRNA transcripts, wherein the 2′-OH group can be replaced by a group selected from H, O—R, R, halogen (e.g., F, Cl, Br), SH, SR, NH2, NHR, and NRR′, or S, wherein R and R′ are independently C1-C6 alkyl. In certain embodiments, the oligonucleotides can have one or more non-natural phosphodiester linkage selected from the group comprising phosphorothioate, methylphosphonate, and a peptide.

Nucleobase-modified building blocks comprise a non-standard nucleobase instead of a standard nucleobase (e.g., adenine, guanine, cytosine, thymine or uracil) such as a uracil or cytosine modified at the 5-position, e.g., 5-methylcytosine, 5-(2-amino) propyluracil, 5-bromouracil, adenines or guanines modified at the 8-position, e.g., 8-bromoguanine, deazapurine nucleobases, e.g., 7-deaza-adenine and O- or N-alkylated nucleobases, e.g., N6 alkyl-adenine. Other examples include the nucleotide substitution of at least one uracil nucleotide with a 5-propynyluracil nucleotide, 5-methyluridine nucleotide, or deoxythymidine nucleotide, at least one cytosine nucleotide with a 5-methylcytosine nucleotide, or at least one adenine or guanine nucleotide with an 8-bromoadenine or 8-bromoguanine nucleotide.

In certain embodiments, the oligonucleotide comprises at least one modified nucleotide. In certain embodiments, the modified nucleotide is chosen from the group of: a 2′-O-methyl modified nucleotide, a nucleotide comprising a 5′phosphorothioate group, and a terminal nucleotide linked to a cholesteryl derivative or dodecanoic acid bisdecylamide group. In certain embodiments, the modified nucleotide is chosen from the group of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide. In certain embodiments, the dsRNA comprises at least one 2′-O-methyl modified nucleotide and at least one nucleotide comprising a 5′phosphorothioate group.

In certain embodiments, the oligonucleotides can comprise one or more locked nucleic acid (LNA) moieties in which the 2′ oxygen is covalently linked to the 4′ carbon of the pentose.

In some embodiments, the 2′ position of a nucleotide is modified. In such cases, a nucleotide with a 2′ modification confers improved stability and extends the half-life of the dsRNA molecule. Non-limiting examples of a modified nucleotide include: a 2′-O-methyl modified nucleotide, a 5′-phosphorothioate group modified nucleotide, a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, a non-natural base comprising nucleotide, and a terminal nucleotide linked to a cholesteryl derivative or dodecanoic acid bisdecylamide group. In some embodiments, the modified nucleotide is a 2′-O-methyl modified nucleotide.

In some embodiments, the modified nucleotide comprises a conformationally constrained nucleotide. In some embodiments, the conformationally constrained nucleotide is a Locked Nucleic Acid (LNA). In some embodiments, LNA base(s) improves the stability of an oligonucleotide (e.g., a siRNA targeting Titin isoform N2B) and increases the binding affinity to RNA (e.g., the Cyclin D2 transcript).

In some embodiments, an oligonucleotide (e.g., a siRNA) includes at least one modification at a phosphate linkage. In some embodiments, an oligonucleotide includes at least one (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 or more) modified phosphate linkage(s).

Non-limiting examples of modified phosphate linkages include: phosphorothioate linkages, methylphosphonate linkages, ethylphosphonate linkages, boranophosphate linkages, sulfonamide, carbonylamide, phosphorodiamidate, phosphorodiamidate linkages comprising a positively charged side group, phosphorodithioates, aminoethylglycine, phosphotriesters, aminoalkylphosphotriesters; 3′-alkylene phosphonates; 5′-alkylene phosphonates, chiral phosphonates, phosphinates, 3′-amino phosphoramidate, aminoalkylphosphoramidates, thionophosphoramidates; thionoalkyl-phosphonates, thionoalkylphosphotriesters, selenophosphates, 2′-5′ linked boranophosphonate analogs, linkages having inverted polarity, abasic linkages, short chain alkyl linkages, cycloalkyl internucleoside linkages, mixed heteroatom and alkyl or cycloalkyl internucleoside linkages, short chain heteroatomic or heterocyclic internucleoside linkages with siloxane backbones, sulfide, sulfoxide, sulfone, formacetyl linkages, thioformacetyl linkages, methylene formacetyl linkages, thioformacetyl linkages, riboacetyl linkages, alkene linkages, sulfamate backbones, methyleneimino linkages, methylenehydrazino linkages, sulfonate linkages, and amide linkages (as described in WO 2012/145729, which is herein incorporated by reference in its entirety).

In some embodiments, the phosphate linkage includes a phosphorothioate bond. In such cases, the phosphorothioate (PS) bond substitutes a sulfur atom for a non-bridging oxygen in the phosphate backbone of an oligo. In some embodiments, the phosphorothioate bond modification renders the internucleotide linkage resistant to nuclease degradation. Non-limiting examples of phosphorothioate (PS) bonds are as described in as described in WO 2012/145729, which is herein incorporated by reference in its entirety.

In some embodiments, the phosphate linkage includes a boranophosphate linkage. In such cases, the boranophosphate linkage is more resistant to degradation by endo- and exonucleases relative to a normal phosphodiester linkage.

In some embodiments, the sense region, antisense region, or both the sense and antisense strands comprise at least one (e.g., at least two, at least three, or at least four) non-natural phosphodiester linkage. In some embodiments, the sense strand, the antisense strand, or both comprise at least five (e.g., at least six, at least seven, at least eight, or at least nine) non-natural phosphodiester linkages. In some embodiments, the sense strand, the antisense strand, or both comprise at ten (e.g., at least 1, at least 12, at least 13, or at least 14 or more) non-natural phosphodiester linkages.

In some embodiments, a modified nucleotide includes a modification at the ribose ring. Non-limiting examples of modified nucleic acids with modification at the ribose ring include: 6-membered (hexitol (HNA), cyclohexenic (CeNA), and altritol (ANA)); 7-membered rings (oxepanic nucleic acid (ONA)); bicyclic (locked nucleic acids (LNA) or 2′-deoxymethanocarbanucleosides (MCs)); tricyclic (tricyclo-DNA (tc-DNA)), and acyclic (unlocked nucleic acid (UNA)) derivatives). Modified nucleic acids with modifications at the ribose ring can protect siRNAs from the action of nucleases. In some embodiments, a modified nucleic acid with modification at the ribose ring is CeNA. In such cases, CeNA's complementary interaction with RNA stabilizes the duplex, increasing the melting point by 1.5° C. per modified base and increases the oligoribonucleotide resistance to degradation. In some embodiments, a modified nucleic acid with modification at the ribose ring is a bicyclic derivatives (e.g., LNA). In such cases, an LNA increases the melting temperature of siRNA by about 2-8° C. per nucleotide due to the extra cycle between 2′ and 4′ carbon, which fixes the 3′ endo ribose conformation. Additional, ribose ring modifications are as described in Chernikov et al., Front. Pharmacol., 10:444 (2019).

In some embodiments, a modified nucleotide includes a modification at the nucleobase. Non-limiting examples of modified nucleotides having a modification at the nucleobase include: pseudouridine, 2′thiouridine, dihydrouridine, 2,4-difluorobenzene, 4-methylbenzimidazole, hypoxanthine, 7-deazaguanin, N2-alkyl-8-oxoguanine, N2-benzyl-guanine, and 2,6-diaminopurine. In such cases, the modified nucleotides having a modification at the nucleobase are designed to increase the thermal stability of the duplex by increasing the efficiency of the formation of hydrogen bonds with complementary nucleotides on the RNA (e.g., the Cyclin D2 transcript).

In some embodiments, a modified nucleotide includes a modification that is a thermally destabilizing modification. In some embodiments, modifications stabilizing the duplex formed by the 3′ end of the antisense strand and 5′ end of the sense strand and, conversely, modifications destabilizing the duplex formed by the 3′ end of the sense strand and 5′ end of the antisense strand can increase the efficiency of the dsRNA molecule by providing favorable duplex thermal asymmetry.

In some embodiments, the oligonucleotide molecule includes at least one thermally destabilizing modification of the duplex within the seed region. Non-limiting examples of dsRNA that include at least one thermally destabilizing modification of the duplex within the seed region are as described in WO 2018/098328A1, which is herein incorporated by reference in its entirety.

In some embodiments, the oligonucleotides can optionally have at least one 3′-overhang sequence having the length of about 1 to about 6 or about 1 to about 3 nucleotides. In one embodiment, the 3′ overhang sequence can comprise two thymidine residues. In one embodiment, these optional overhangs are not included when calculating the percent homology and/or percent identity described herein. Typical siRNA molecules are for example described in WO 02/044321, the content of which is incorporated herein by reference. In certain embodiments, the oligonucleotides can target at least about 10, about 11, about 12, about 13, about 14, about 15, about 16, about 17, about 18, about 19, about 20, about 21, about 22, about 23, about 24, or about 25 nucleotides of SEQ ID NOs: 31 or 32. In preferred embodiments, the oligonucleotides can target positions 7 to 24, positions 6 to 23, or positions 8 to 25 of SEQ ID NOs: 31 or 32. The targeted sequences can comprise nucleotides from both of the adjacent exons (e.g., from exons 50 and 220).

### 5.2.3 Patterns of Modifications

In some embodiments, the oligonucleotide (e.g., the siRNA targeting Titin isoform N2B) includes a pattern of one or more modified nucleotides (including modified phosphate linkages). In such cases, the pattern of one or more modified nucleotides is designed to confer stability and/or increased silencing activity. See, e.g., Chernikov et al., Front. Pharmacol., 10:444 (2019), which describes modifications and patterns of modifications that confer stability and/or increase silencing activity. As noted above, in some instances, the oligonucleotide (e.g., the siRNA) can be degraded in vivo as a result of its cleavage by endonucleases on pyrimidines and exonucleases from both the 3′ and 5′ ends. Therefore, the oligonucleotide (e.g., the siRNA) can include modifications at cleavage sites to improve oligonucleotide nuclease resistance to achieve biological activity in vivo. However, the introduction of certain modifications in the oligonucleotides (e.g., siRNAs) are limited by inhibition of its interfering activity and toxicity. In such cases, one parameter affecting RNAi is not only the number of introduced modifications, but also their location in the duplex. In some embodiments, the introduction of modifications in the oligonucleotides (e.g., the siRNAs) is determined by the balance between the number of modifications sufficient for dsRNA molecule to be non-toxic, while retaining interfering activity and nuclease resistance. In some embodiments, introducing a 2′-O-methyl modified nucleotide into the oligonucleotide can lead to inhibition of RNAi if the dsRNA molecule contains more than two consecutive nucleotides modified to include a 2′-O-methyl modification. In some embodiments, an oligonucleotide (e.g., a siRNA) comprising a 2′-O-methyl modified nucleotide at every second nucleotide does not block RNAi.

In some embodiments, introduction of a 2′-O-methyl modified nucleotide into known cleavages sites preserves the interfering activity of the oligonucleotide, increases nuclease resistance, and provides long-term suppression of Titin isoform N2B. In some embodiments, known cleavage sites include CA, UA, and UG sites. In such cases, introducing a 2′-O-methyl modified nucleotide to these sites protects the oligonucleotide (e.g., the siRNA) from cleavage.

In some embodiments, introduction of a 2′-Fluoro-deoxyadenosine (2′-F) modified nucleotide into known cleavages sites preserves the interfering activity of the oligonucleotide (e.g., the siRNA), increases nuclease resistance, and provides long-term suppression of Titin isoform N2B. In some embodiments, (2′-F) modified nucleotide are incorporated at terminal nucleotides.

In some embodiments, an oligonucleotide (e.g., a siRNA) comprises alternating 2′O-Me and 2′F modifications. In such cases, these oligonucleotides are stable in blood plasma and suppress expression of the target gene.

In some embodiments, introduction of a phosphorothioate (PS) into the oligonucleotide (e.g., the siRNA) preserves the interfering activity of the oligonucleotide, increases nuclease resistance, and provides long-term suppression of Titin isoform N2B. In some embodiments, PS are incorporated at terminal nucleotides.

In some embodiments, an oligonucleotide (e.g., a siRNA) comprises one or more 2′O-Me modifications, one or more 2′F modifications, or more or more PS modification, or any combination thereof. In some embodiments, the optimal introduction of 2′O-Me or 2′F modifications for each position in the siRNA is determined via in vitro analysis in cardiomyocytes (e.g., induced pluripotent stem cell derived cardiomyocytes (iPSC-CM)), or any other suitable system.

In some embodiments, an oligonucleotide (e.g., a siRNA) comprises PS modifications of the 5′ ends in the N-acetylgalactosamine conjugate. In such cases, PS modifications of the 5′ ends in the N-acetylgalactosamine conjugate increase the duration and efficiency of the siRNA's inhibitory effect. In some embodiments, conjugates are fully modified at the 2′ positions and stabilized by PS modifications at both the 3′ and 5′ ends of the siRNA.

In embodiments, suitable modified sequences include modifications to SEQ ID NOs: 1, 3, 5, 7, 9, 11, 13, 15, 17, 19, 21, 23, 25, 27, 29. In embodiments, the modified sequences may be a sense strand, or hybridized with another nucleic acid strand, such as an antisense strand to form a duplex. In embodiments, the sequences are characterized as:

TABLE 3
Sense Strands with modification

| | | Corresponding |
|---|---|---|
| | | un-modified |
| SEQ ID | | sequence |
| NO: | Sequence with Modifications | (SEQ ID NO:) |
| 54 | 5′- | 1 |
| | [fU][Ps][mA][Ps][fA][mC][fU][mG][fU][mG][fA][mC][fA][m | |
| | G][fU][mG][Ps][fA][Ps][3-CholTEG] | |
| 55 | 5′- | 3 |
| | [fA][Ps][mA][Ps][fC][mU][fG][mU][fG][mA][fC][mA][fG][m | |
| | U][fG][mC][Ps][fA][Ps][3-CholTEG] | |
| 56 | 5′- | 5 |
| | [[A][Ps][mC][Ps][fU][mG][fU][mG][fA][mC][fA][mG][fU][m | |
| | G][fC][mC][Ps][fA][Ps][3-CholTEG] | |
| 57 | 5′- | 7 |
| | [fC][Ps][mU][Ps][fG][mU][fG][mA][fC][mA][fG][mU][fG][m | |
| | C][fC][mU][Ps][fA][Ps][3-CholTEG] | |
| 58 | 5′- | 9 |
| | [fU][Ps][mG][Ps][fU][mG][fA][mC][fA][mG][fU][mG][fC][m | |
| | C][fU][mG][Ps][fA][Ps][3-CholTEG] | |
| 59 | 5′- | 11 |
| | [fG][Ps][mU][Ps][fG][mA][fC][mA][fG][mU][fG][mC][fC][m | |
| | U][fG][mG][Ps][fA][Ps][3-CholTEG] | |
| 60 | 5′- | 13 |
| | [fU][Ps][mG][Ps][fA][mC][fA][mG][fU][mG][fC][mC][fU][m | |
| | G][fG][mA][Ps][fA][Ps][3-CholTEG] | |
| 61 | 5′- | 15 |
| | [fG][Ps][mA][Ps][fC][mA][fG][mU][fG][mC][fC][mU][fG][m | |
| | G][fA][mG][Ps][fA][Ps][3-CholTEG] | |
| 62 | 5′- | 17 |
| | [fA][Ps][mC][Ps][fA][mG][fU][mG][fC][mC][fU][mG][fG][m | |
| | A][fG][mG][Ps][fA][Ps][3-CholTEG] | |
| 63 | 5′- | 19 |
| | [fC][Ps][mA][Ps][fG][mU][fG][mC][fC][mU][fG][mG][fA][m | |
| | G][fG][mU][Ps][fA][Ps][3-CholTEG] | |
| 64 | 5′- | 21 |
| | [fA][Ps][mG][Ps][fU][mG][fC][mC][fU][mG][fG][mA][fG][m | |
| | G][fU][mG][Ps][fA][Ps][3-CholTEG] | |
| 65 | 5′- | 23 |
| | [fG][Ps][mU][Ps][fG][mC][fC][mU][fG][mG][fA][mG][fG][m | |
| | U][fG][mA][Ps][fA][Ps][3-CholTEG] | |
| 66 | 5′- | 25 |
| | [fU][Ps][mG][Ps][fC][mC][fU][mG][fG][mA][fG][mG][fU][m | |
| | G][fA][mA][Ps][fA][Ps][3-CholTEG] | |
| 67 | 5′- | 27 |
| | [fG][Ps][mC][Ps][fC][mU][fG][mG][fA][mG][fG][mU][fG][m | |
| | A][fA][mA][Ps][fA][Ps][3-CholTEG] | |
| 68 | 5′- | 29 |
| | [fC][Ps][mC][Ps][fU][mG][fG][mA][fG][mG][fU][mG][fA][m | |
| | A][fA][mA][Ps][fA][Ps][3-CholTEG] | |

As shown in Table 3 above, “m” refers to 2′-OMe modification in each instance, “f” refers to −2′Fluoro modification in each instance, “Ps” refers to a Phosphorothioate linkage in each instance, Chol-TEG refers to a cholesterol modification in each instance. The nucleic acid strands are written 5′ to 3′ in the paragraph above. In embodiments, the positioning of the Chol-TEG provides a suitable alteration for self-delivery of the molecules to a subject in need thereof. In embodiments, the present disclosure includes fully modified nucleic acid sequences, wherein each nucleotide is characterized as modified. In embodiments, the present disclosure includes partially modified nucleic acid sequences, wherein 1-5, 1-10, or 1-14 nucleotides is characterized as modified. In embodiments, the present disclosure includes non-modified nucleic acid sequences such as depicted in SEQ ID NO: 1, 3, 5, 7, 9, 11, 13, 15, 17, 19, 21, 23, 25, 27, and 29.

In embodiments, suitable modified sequences include modifications to SEQ ID NOs: 2, 4, 6, 8, 10, 12, 14, 16, 20, 22, 24, 26, 28, and 30. In embodiments, the modified sequences may be an antisense strand, or hybridized with another nucleic acid strand, such as a sense strand, to form a duplex. In embodiments, the sequences are characterized as:

TABLE 4
Anti-sense Strands with modification

| SEQ ID | |
|---|---|
| NO: | Sequence with Modifications |
| 37 | 5′-[5Phos][mU][Ps][fC][Ps][mA][fC][mU][fG][mU][fC][mA][fC][mA][fG][mU] |
| | [fU][Ps][mA][Ps][fG][Ps][mU][Ps][fG][Ps][mU][Ps][fG] |
| 38 | 5′-[5Phos][mU][Ps][fG][Ps][mC][fA][mC][fU][mG][fU][mC][fA][mC][fA][mG] |
| | [fU][Ps][mU][Ps][fA][Ps][mG][Ps][fU][Ps][mG][Ps][fU] |
| 39 | 5′-[5Phos][mU][Ps][fG][Ps][mG][fC][mA][fC][mU][fG][mU][fC][mA][fC][mA] |
| | [fG][Ps][mU][Ps][fU][Ps][mA][Ps][fG][Ps][mU][Ps][fG] |
| 40 | 5′-[5Phos][mU][Ps][fA][Ps][mG][fG][mC][fA][mC][fU][mG][fU][mC][fA][mC] |
| | [fA][Ps][mG][Ps][fU][Ps][mU][Ps][fA][Ps][mG][Ps][fU] |
| 41 | 5′-[5Phos][mU][Ps][fC][Ps][mA][fG][mG][fC][mA][fC][mU][fG][mU][fC][mA] |
| | [fC][Ps][mA][Ps][fG][Ps][mU][Ps][fU][Ps][mA][Ps][fG] |
| 42 | 5′-[5Phos][mU][Ps][fC][Ps][mC][fA][mG][fG][mC][fA][mC][fU][mG][fU][mC] |
| | [fA][Ps][mC][Ps][fA][Ps][mG][Ps][fU][Ps][mU][Ps][fA] |
| 43 | 5′-[5Phos][mU][Ps][fU][Ps][mC][fC][mA][fG][mG][fC][mA][fC][mU][fG][mU] |
| | [fC][Ps][mA][Ps][fC][Ps][mA][Ps][fG][Ps][mU][Ps][fU] |
| 44 | 5′-[5Phos][mU][Ps][fC][Ps][mC][fU][mC][fC][mA][fG][mG][fC][mA][fC][mU] |
| | [fG][Ps][mU][Ps][fC][Ps][mA][Ps][fC][Ps][mA][Ps][fG] |
| 45 | 5′-[5Phos][mU][Ps][fA][Ps][mC][fC][mU][fC][mC][fA][mG][fG][mC][fA][mC] |
| | [fU][Ps][mG][Ps][fU][Ps][mC][Ps][fA][Ps][mC][Ps][fA] |
| 46 | 5′-[5Phos][mU][Ps][fC][Ps][mA][fC][mC][fU][mC][fC][mA][fG][mG][fC][mA] |
| | [fC][Ps][mU][Ps][fG][Ps][mU][Ps][fC][Ps][mA][Ps][fC] |
| 47 | 5′-[5Phos][mU][Ps][fU][Ps][mC][fA][mC][fC][mU][fC][mC][fA][mG][fG][mC] |
| | [fA][Ps][mC][Ps][fU][Ps][mG][Ps][fU][Ps][mC][Ps][fA] |
| 48 | 5′-[5Phos][mU][Ps][fU][Ps][mU][fC][mA][fC][mC][fU][mC][fC][mA][fG][mG] |
| | [fC][Ps][mA][Ps][fC][Ps][mU][Ps][fG][Ps][mU][Ps][fC] |
| 49 | 5′-[5Phos][mU][Ps][fU][Ps][mU][fU][mC][fA][mC][fC][mU][fC][mC][fA][mG] |
| | [fG][Ps][mC][Ps][fA][Ps][mC][Ps][fU][Ps][mG][Ps][fU] |
| 50 | 5′-[5Phos][mU][Ps][fU][Ps][mU][fU][mU][fC][mA][fC][mC][fU][mC][fC][mA] |
| | [fG][Ps][mG][Ps][fC][Ps][mA][Ps][fC][Ps][mU][Ps][fG] |

As shown in Table 4 above, “m” refers to 2′-OMe modification in each instance, “f” refers to −2′Fluoro modification in each instance, “Ps” refers to a Phosphorothioate linkage in each instance. The nucleic acid strands are written 5′ to 3′ in the paragraph above. In embodiments, the present disclosure includes fully modified nucleic acid sequences, wherein each nucleotide is characterized as modified. In embodiments, the present disclosure includes partially modified nucleic acid sequences, wherein 1-5, 1-10, or 1-19 nucleotides is characterized as modified.

### 5.2.4 Conjugation

In some embodiments, the oligonucleotide may be conjugated to a non-nucleic acid moiety. Non-limiting examples of a non-nucleic acid moiety include a fatty acid, a lipid, a peptide, a protein, an antibody, or a nanoparticle.

In some cases, where an oligonucleotide is covalently linked to a non-nucleotide moiety the resulting molecule is referred to as a conjugate. In some embodiments, conjugation of an oligonucleotide of to one or more non-nucleotide moieties may improve the pharmacology of the oligonucleotide, for example, by affecting the activity, cellular distribution, cellular uptake, or stability of the oligonucleotide-based therapeutic. In some embodiments, the non-nucleotide moieties modify or enhance the pharmacokinetic properties of the oligonucleotide by improving cellular distribution, bioavailability, metabolism, excretion, permeability, and/or cellular uptake of the oligonucleotide-based therapeutic. In certain embodiments, the non-nucleotide moieties may target the oligonucleotide-based therapeutic to the heart. In other embodiments, the non-nucleotide moieties reduce the activity of the oligonucleotide in non-target cell types, tissues, or organs, e.g., off target activity or activity in non-target cell types, tissues, or organs.

In some embodiments, oligonucleotides are conjugated to conjugates where conjugates are as described in WO 2013/033230, which is herein incorporated by reference in its entirety.

Conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO 1, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., as described in WO 2014/179620). In some embodiments, the oligonucleotide-based therapeutic is conjugated to a N-acetylgalactosamine (“a N-acetylgalactosamine conjugate”).

In some embodiments, the non-nucleotide moiety is a cholesterol moiety. In some embodiments, the cholesterol moiety and the mechanism and/or features by which the cholesterol moiety is attached to the oligonucleotide is as described in U.S. Pat. No. 10,933,081B2, which is herein incorporated by reference in its entirety. In some embodiments, the cholesterol is attached to the 3′ terminal nucleotide (either the sense strand, antisense strand, or both) of the oligonucleotide. In some embodiments, the cholesterol is attached to the 5′ terminal nucleotide (either the sense strand, antisense strand, or both) of the oligonucleotide. In some embodiments, the cholesterol is attached to an internal nucleotide of the oligonucleotide.

In some embodiments, the non-nucleotide moiety (conjugate moiety) is selected from the group consisting of carbohydrates, cell surface receptor ligands, drug substances, hormones, lipophilic substances, polymers, proteins, peptides, toxins (e.g., bacterial toxins), vitamins, viral proteins (e.g., capsids), and combinations thereof.

In some embodiments, an oligonucleotide-based therapeutic is conjugated to at least two (e.g., at least three, at least, at least four, at least five, at least six, at least seven, at least eight, at least nine, or at least ten) non-nucleotide moieties.

In some embodiments, an oligonucleotide-based therapeutic is conjugated to a cardiac targeting peptide. Non-limiting examples of cardiac-targeting peptides are as described in Kim et al. (Mol. Ther. Nucl. Acids, P1024-1032 (2021)), which is herein incorporated by reference in its entirety.

In some embodiments, an oligonucleotide-based therapeutic is activated. In such cases, an activated oligonucleotide refers to an oligonucleotide-based therapeutic that is covalently linked to a functional moiety that permits covalent linkage of the oligonucleotide to one or more conjugated moieties. In some embodiments, a functional moiety will comprise a chemical group that is capable of covalently bonding to the oligonucleotide-based therapeutic via, for example, a 3′-hydroxyl group or the exocyclic NH2 group of the adenine base, a spacer that can be hydrophilic and a terminal group that is capable of binding to a conjugated moiety (e.g., an amino, sulfhydryl or hydroxyl group). In some embodiments, this terminal group is not protected, for example, is an NH2 group. In other embodiments, the terminal group is protected, for example, by any suitable protecting group such as those described in “Protective Groups in Organic Synthesis” by Theodora W. Greene and Peter G. M. Wuts, 3rd edition (John Wiley & Sons, 1999), which is hereby incorporated by reference.

In some embodiments, the oligonucleotide-based therapeutic provided herein are functionalized at the 5′ end in order to allow covalent attachment of the conjugated moiety to the 5′ end of the oligonucleotide (e.g., on the 5′ end of the antisense strand of a dsRNA molecule). In some embodiments, the oligonucleotides provided herein can be functionalized at the 3′ end (e.g., on the 3′ end of the antisense strand of a dsRNA molecule). In some embodiments, oligonucleotides provided herein can be functionalized along the backbone or on the heterocyclic base moiety. In yet other embodiments, the oligonucleotides provided herein can be functionalized at more than one position independently selected from the 5′ end, the 3′ end, the backbone, and the base.

In some embodiments, activated oligonucleotides are synthesized by incorporating during the synthesis one or more monomers that is covalently attached to a functional moiety. In some embodiments, activated oligonucleotides are synthesized with monomers that have not been functionalized, and the oligonucleotide-based therapeutic is functionalized upon completion of synthesis.

In some embodiments, an oligonucleotide is conjugated to a ligand. In some embodiments, the oligonucleotide-based therapeutic is conjugated to one or more ligands described in WO 2018/044350, which is herein incorporated by reference in its entirety. Methods for linking ligands to the oligonucleotide-based therapeutic are known in the art, for example, as described in WO 2018/044350.

In some embodiments where the oligonucleotide comprises a dsRNA molecule, the dsRNA molecule can be conjugated to a non-ligand molecule. For example, non-ligand molecules have been conjugated to dsRNAs in order to enhance the activity, cellular distribution or cellular uptake of the dsRNA, and procedures for performing such conjugations are available in the scientific literature. Non-limiting examples on non-ligand moieties are as described in WO 2010/048228, which is herein incorporated by reference in its entirety.

In some embodiments, typical conjugation protocols involve the synthesis of dsRNAs bearing an amino linker at one or more positions of the sequence. In such cases, the amino group is then reacted with the molecule being conjugated using appropriate coupling or activating reagents. In some embodiments, the conjugation reaction may be performed either with the dsRNA molecule still bound to the solid support or following cleavage of the dsRNA molecule in solution phase.

### 5.3 Pharmaceutical Composition, Dose, and Dose Schedule

In another aspect, this disclosure provides a pharmaceutical composition that can specifically target the Titin N2B isoform mRNA expression with little or no effect on Titin N2BA isoform expression for the use in medicine. In certain embodiments, the pharmaceutical composition comprises an effective amount of an oligonucleotide as described herein.

In some embodiments, the pharmaceutical composition is formulated as a delivery system for the oligonucleotide, wherein the oligonucleotide can be used in a liposome, nanocapsule, microemulsion droplet, or other biosurfactant-based pharmaceutical delivery system with the oligonucleotide encapsulated therein.

In some embodiments, the pharmaceutical compositions are formulated as an orally-consumable product, such as, for example a food item, capsule, pill, or drinkable liquid. An orally deliverable pharmaceutical is any physiologically active substance delivered via initial absorption in the gastrointestinal tract or into the mucus membranes of the mouth. The topic compositions can also be formulated as a solution that can be administered via, for example, injection, which includes intravenously, intraperitoneally, intramuscularly, intrathecally, or subcutaneously. In other embodiments, the subject compositions are formulated to be administered via the skin through a patch or directly onto the skin for local or systemic effects. The compositions can be administered sublingually, buccally, rectally, or vaginally. Furthermore, the compositions can be sprayed into the nose for absorption through the nasal membrane, nebulized, inhaled via the mouth or nose, or administered in the eye or ear.

Orally consumable products according to the present disclosure are any preparations or compositions suitable for consumption, for nutrition, for oral hygiene, or for pleasure, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes). While an orally-deliverable pharmaceutical can be formulated into an orally consumable product, and an orally consumable product can comprise an orally deliverable pharmaceutical, the two terms are not meant to be used interchangeably herein.

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state; the orally consumable products according to the present disclosure therefore also include casings, coatings, or other encapsulations that are intended to be swallowed together with the product or for which swallowing is to be anticipated.

In some embodiments, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally-consumable product.

In some embodiments, the orally-consumable product according to the present disclosure can comprise one or more formulations intended for nutrition or pleasure. These particularly include baking products (e.g., bread, dry biscuits, cake, cookies, and other pastries), sweets (e.g., chocolates, chocolate bar products, other bar products, fruit gum, coated tablets, hard caramels, toffees and caramels, and chewing gum), alcoholic or non-alcoholic beverages (e.g., cocoa, coffee, green tea, black tea, black or green tea beverages enriched with extracts of green or black tea, Rooibos tea, other herbal teas, fruit-containing lemonades, isotonic beverages, soft drinks, nectars, fruit and vegetable juices, and fruit or vegetable juice preparations), instant beverages (e.g., instant cocoa beverages, instant tea beverages, and instant coffee beverages), meat products (e.g., ham, fresh or raw sausage preparations, and seasoned or marinated fresh meat or salted meat products), eggs or egg products (e.g., dried whole egg, egg white, and egg yolk), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, whey, butter, buttermilk, and partly or wholly hydrolyzed products containing milk proteins), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sauces, and fruit fillings), vegetable preparations (e.g., ketchup, sauces, dried vegetables, deep-freeze vegetables, pre-cooked vegetables, and boiled vegetables), snack articles (e.g., baked or fried potato chips (crisps) or potato dough products and extrudates on the basis of maize or peanuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening or whitening beverages or other food). The present compositions may also serve as semi-finished products for the production of other compositions intended for nutrition or pleasure.

In some embodiments, the subject composition can further comprise one or more pharmaceutically acceptable carriers, and/or excipients, and can be formulated into preparations, for example, solid, semi-solid, liquid, or gaseous forms, such as tablets, capsules, powders, granules, ointments, solutions, suppositories, injections, inhalants, and aerosols.

The term “pharmaceutically acceptable” as used herein means compatible with the other ingredients of a pharmaceutical composition and not deleterious to the recipient thereof.

In some embodiments, carriers and/or excipients according the subject disclosure can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g., carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target oligonucleotide, carrier or excipient use in the subject compositions may be contemplated.

In some embodiments, the pharmaceutical composition can be made into aerosol formulations so that, for example, it can be nebulized or inhaled. Suitable pharmaceutical formulations for administration in the form of aerosols or sprays are, for example, powders, particles, solutions, suspensions or emulsions. Formulations for oral or nasal aerosol or inhalation administration may also be formulated with carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In some embodiments, the adjuvant composition can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

In some embodiments, the pharmaceutical composition can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

Further components can be added to the compositions as needed such as, for example, buffers, carriers, viscosity modifiers, preservatives, flavorings, dyes and other ingredients specific for an intended use. One skilled in this art will recognize that the above description is illustrative rather than exhaustive. Indeed, many additional formulations techniques and pharmaceutically-acceptable excipients and carrier solutions suitable for particular modes of administration are available.

### 5.3.1 Routes of Administration

Any of the pharmaceutical compositions provided herein or any of the oligonucleotides (e.g., siRNAs) provided herein can be administered in a number of ways depending upon whether local or systemic treatment is desired and upon the area to be treated. Non-limiting examples of drug delivery systems for oligonucleotides (e.g., siRNAs) include those described in Paunovska et al., Nat. Rev. Gen., 40 (2022). As described in Paunovska, oligonucleotides can be delivered using synthetic delivery vehicles (e.g., lipids, lipid-based nanoparticles, polymers, and polymer-based nanoparticles). Selection of delivery vehicles depend on factors including, without limitation, therapeutic-related factors (e.g., size, stability, among others), tissue location, duration of desired expression, whether targeting is active or passive or active tissue targeting, among other criteria).

In some embodiments, administration can be (a) oral; (b) pulmonary, e.g., by inhalation or insufflation of powders or aerosols, including by nebulizer; intratracheal, intranasal, (c) topical including epidermal, transdermal, ophthalmic and to mucous membranes including vaginal and rectal delivery; or (d) parenteral including intravenous, intraarterial, subcutaneous, intraperitoneal or intramuscular injection or infusion; or intracranial, e.g., intrathecal, intra-cerebroventricular, or intraventricular, administration. In some embodiments, the oligonucleotides (or pharmaceutical compositions) are administered intravenously, intraperitoneally, orally, topically, or as a bolus injection or administered directly into the target organ. In some embodiments, the oligonucleotides (or pharmaceutical compositions) are administered intracardially or intraventricularly as a bolus injection. In some embodiments, the oligonucleotides (or pharmaceutical compositions) are administered subcutaneously. In some embodiments, the oligonucleotides (or pharmaceutical compositions) are administered orally.

In some embodiments, pharmaceutical compositions and formulations for topical administration can include transdermal patches, ointments, lotions, creams, gels, drops, sprays, suppositories, liquids and powders. Conventional pharmaceutical carriers, aqueous, powder or oily bases, thickeners and the like may be necessary or desirable. Examples of topical formulations include those in which any of the oligonucleotide-based therapeutics provided herein are in admixture with a topical delivery agent such as lipids, liposomes, fatty acids, fatty acid esters, steroids, chelating agents and surfactants. Compositions and formulations for oral administration include but are not limited to powders or granules, microparticulates, nanoparticulates, suspensions or solutions in water or non-aqueous media, capsules, gel capsules, sachets, tablets or minitablets. Compositions and formulations for parenteral, intrathecal, intra-cerebroventricular, or intraventricular administration can include sterile aqueous solutions which can also contain buffers, diluents and other suitable additives such as, but not limited to, penetration enhancers, carrier compounds and other pharmaceutically acceptable carriers or excipients.

In some embodiments, pharmaceutical compositions of the present disclosure include, but are not limited to, solutions, emulsions, and liposome-containing formulations. These compositions may be generated from a variety of components that include, but are not limited to, preformed liquids, self-emulsifying solids and self-emulsifying semisolids. Delivery of oligonucleotide-based therapeutic to the target tissue can be enhanced by carrier-mediated delivery including, but not limited to, cationic liposomes, cyclodextrins, porphyrin derivatives, branched chain dendrimers, polyethylenimine polymers, nanoparticles and microspheres.

The pharmaceutical formulations of the present disclosure, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

In some embodiments, for parenteral, subcutaneous, intradermal, or topical administration the formulation can include a sterile diluent, buffers, regulators of tonicity and antibacterials. In some embodiments, the oligonucleotide-based therapeutics can be prepared with carriers that protect against degradation or immediate elimination from the body, including implants or microcapsules with controlled release properties. For intravenous administration the carriers can be physiological saline or phosphate buffered saline. International Publication No. WO 2007/031091 further provides suitable pharmaceutically acceptable diluent, carrier and adjuvants, which is herein incorporated by reference in its entirety.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Suitable topical formulations include those in which the oligonucleotide-based therapeutics featured in the present disclosure are in admixture with a topical delivery agent such as lipids, liposomes, fatty acids, fatty acid esters, steroids, chelating agents and surfactants.

In some embodiments, the oligonucleotides (e.g., siRNAs) are formulated in lipid formulations. Suitable lipids and liposomes include neutral (e.g., dioleoylphosphatidyl DOPE ethanolamine, dimyristoylphosphatidyl choline DMPC, distearoylphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA).

In some embodiments, oligonucleotides (e.g., siRNAs) provided herein may be encapsulated within liposomes or may form complexes thereto, in particular to cationic liposomes. Alternatively, oligonucleotides may be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C1-10 alkyl ester (e.g., isopropylmyristate IPM), monoglyceride, diglyceride or pharmaceutically acceptable salt thereof.

In some embodiments, an oligonucleotide (e.g., a siRNA) as provided herein for inhibiting or reducing expression of TTN isoform N2B is formulated in a lipid formulation. In some embodiments, a lipid formulation is selected from: a LNP formulation, a LNPO1 formulation, a XTC-SNALP formulation, or a SNALP formulation.

Non-limiting examples of lipid formation are those described in WO 2010/048228A1, which is herein incorporated by reference in its entirety.

### 5.3.2 Dosage and Dosing Schedules

In some embodiments, an oligonucleotide (e.g., siRNA) is administered to a patient at dosage of about 0.01 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 6.0 mg/kg, about 7.0 mg/kg, about 7.5 mg/kg, about 8.0 mg/kg, about 8.5 mg/kg, about 9.0 mg/kg, about 9.5 mg/kg, or about 10 mg/kg. In some embodiments, an oligonucleotide (e.g., siRNA) is administered to a patient at about 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 44 mg/kg, 45 mg/kg, 46 mg/kg, 47 mg/kg, 48 mg/kg, 49 mg/kg, or 50 mg/kg.

In some embodiments, an oligonucleotide (e.g., a siRNA) is administered to a patient at a dosage between 0.01 and 0.2 mg/kg. For example, the dsRNA is administered at a dose of about 0.01 mg/kg, 0.02 mg/kg, 0.3 mg/kg, 0.04 mg/kg, 0.05 mg/kg, 0.06 mg/kg, 0.07 mg/kg 0.08 mg/kg 0.09 mg/kg, 0.10 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, or 0.20 mg/kg.

In some embodiments, the oligonucleotides (e.g., siRNAs) may be administered once daily, or the oligonucleotide may be administered as two, three, or more sub-doses at appropriate intervals throughout the day or even using continuous infusion or delivery through a controlled release formulation. In that case, the oligonucleotide contained in each sub-dose must be correspondingly smaller in order to achieve the total daily dosage. In some embodiments, the dosage unit can also be compounded for delivery over several days, e.g., using a conventional sustained release formulation which provides sustained release of the oligonucleotide-based therapeutic over a several day period. Sustained release formulations are well known in the art and are particularly useful for delivery of agents at a particular site, such as could be used with the agents of the present disclosure. In this embodiment, the dosage unit contains a corresponding multiple of the daily dose. Non-limiting examples of dose and dosing schedules for oligonucleotide-based therapeutics are as described in WO 2010/048228, which is herein incorporated by reference in its entirety.

In some embodiments, the effect of a single dose of the oligonucleotide on TTN isoform N2B levels is long lasting, such that subsequent doses are administered at not more than 3, 4, or 5 day intervals, or at not more than 1, 2, 3, or 4 week intervals, or at not more than 5, 6, 7, 8, 9, or 10 week intervals.

In some embodiments, at least 40%, for example at least 45%, at least 50%, at least 55%, at least 60%, at least 65%, at least 70%, at least 75%, or at least 80% of the antisense strand of the siRNA is present in vivo, for example in a cardiomyocyte, at day 2, 3, 4, 5, 6, 7, 8, 9, 10, 11, 12, 13, 14, 15, 16, 17, 18, 19, 20, 21, 22, 23, 24, or 25 after in vivo administration.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of an oligonucleotide-based therapeutic can include a single treatment or a series of treatments. Estimates of effective dosages and in vivo half-lives for the individual oligonucleotide (e.g., siRNA) provided herein can be made using conventional methodologies or on the basis of in vivo testing using an appropriate animal model, as known in the art.

In some embodiments, the data obtained from cell culture assays and animal studies can be used in formulating a range of dosage for use in humans. The dosage of compositions provided herein lies generally within a range of circulating concentrations that include the ED50 with little or no toxicity. The dosage may vary within this range depending upon the dosage form employed and the route of administration utilized. For any compound used in the methods provided herein, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of TTN isoform N2B mRNA or protein) that includes the IC50 (i.e., the concentration of the test compound which achieves a half-maximal inhibition of symptoms) as determined in cell culture. In some embodiments, such information can be used to more accurately determine useful doses in humans. In such cases, levels in plasma may be measured, for example, by high performance liquid chromatography.

In some embodiments, the oligonucleotide (e.g., siRNA) provided herein can be administered in combination with other known agents effective in treatment of pathological processes mediated by target gene expression. In such cases, the administering physician can adjust the amount and timing of the oligonucleotides (e.g., siRNAs) administration on the basis of results observed using standard measures of efficacy known in the art or described herein.

### 5.3.3 Additional Methods for Delivering Oligonucleotide that Suppresses Titin Isoform N2B

In some embodiments, oligonucleotide (e.g., siRNA) are administered to a patient or contacted with a cell (e.g., a cardiomyocyte) using a vector. In some embodiments, the vector is an expression vector (e.g., a DNA plasmid vector) or a viral vectors. In some embodiments, the vector is a viral vector. In cases where the oligonucleotides (e.g., siRNAs) is delivered using a vector, appropriate regulatory elements (e.g., promoters, enhancers, polyA signals, or any combination thereof) are used to drive expression of the oligonucleotides (e.g., siRNAs).

Any viral vector capable of accepting the coding sequences for the oligonucleotides (e.g., siRNAs) to be expressed can be used. In some embodiments, the tropism of viral vectors can be modified by pseudotyping the vectors with envelope proteins or other surface antigens from other viruses, or by substituting different viral capsid proteins, as appropriate. Non-limiting examples of viral vectors include: lentivirus, retrovirus, gammaretroviruses, adeno-associated virus, adenovirus, helper-dependent adenovirus, sendai virus, or a baculovirus.

In some embodiments, lentiviral vectors are used to deliver the oligonucleotides (e.g., siRNAs). In such cases, the lentiviral vector can be pseudotyped with surface proteins from vesicular stomatitis virus (VSV), rabies, Ebola, Mokola, and the like.

In some embodiments, AAV vectors are used to deliver the oligonucleotides (e.g., siRNA). In such cases, a particular AAV capsid protein serotype can be selected based on its ability to infect the heart (e.g., cardiomyocytes within the heart). In some embodiments, AAV serotype can be selected based, in part, on the AAV expression as performed in Zincarelli et al., Mol. Ther. 16 (6): 1073-1080 (2008), which is herein incorporated by reference in its entirety. In some embodiments, an AAV9 capsid is used to target the oligonucleotides (e.g., siRNAs) to the heart. In some embodiments, an AA1, AAV4, AAV6, or AAV8 are used to target the oligonucleotides (e.g., siRNAs) to the heart. Techniques for constructing AAV vectors which express different capsid protein serotypes are within the skill in the art; see, e.g., Rabinowitz et al., J. Virol. 76:791-801 (2002), which is herein incorporated by reference in its entirety.

Selection of recombinant viral vectors suitable for use in the present disclosure, methods for inserting nucleic acid sequences for expressing the dsRNA into the vector, and methods of delivering the viral vector to the cells of interest are within the skill in the art. See, for example, Dornburg R (1995), Gene Therap. 2:301-310; Eglitis M A (1988), Biotechniques 6:608-614; Miller A D (1990), Hum Gene Therap. 1:5-14; Anderson W F (1998), Nature 392:25-30; and Rubinson D A et al., Nat. Genet. 33:401-406, the entire disclosures of which are herein incorporated by reference. Viral vectors can be derived from AV and AAV. In one embodiment, the oligonucleotide (e.g., siRNA) featured in the present disclosure is expressed as two separate, complementary single-stranded RNA molecules from a recombinant AAV vector having, for example, either the U6 or HI RNA promoters, or the cytomegalovirus (CMV) promoter. A suitable AV vector for expressing the dsRNA disclosed herein, a method for constructing the recombinant AV vector, and a method for delivering the vector into target cells, are described in Xia H et al. (2002), Nat. Biotech. 20:1006-1010.

Suitable AAV vectors for expressing the oligonucleotide-based therapeutic featured in the present disclosure, methods for constructing the recombinant AV (or AAV) vector, and methods for delivering the vectors into target cells are described in Samulski R et al. (1987), J. Virol. 61:3096-3101; Fisher K J et al. (1996), J. Virol, 70:520-532; Samulski R et al. (1989), J. Virol. 63:3822-3826; U.S. Pat. Nos. 5,252,479; 5,139,941; International Patent Application No. WO 94/13788; and International Patent Application No. WO 93/24641, the entire disclosures of which are herein incorporated by reference. The promoter driving expression of an oligonucleotide-based therapeutic in either a DNA plasmid or viral vector featured in the present disclosure may be a eukaryotic RNA polymerase I {e.g., ribosomal RNA promoter), RNA polymerase II (e.g., CMV early promoter or actin promoter or Ul snRNA promoter) or generally RNA polymerase III promoter (e.g., U6 snRNA or 7SK RNA promoter) or a prokaryotic promoter, for example the T7 promoter, provided the expression plasmid also encodes T7 RNA polymerase required for transcription from a T7 promoter. The promoter can also direct transgene expression to the pancreas (see, e.g., the insulin regulatory sequence for pancreas (Bucchini et al., 1986, Proc. Natl. Acad. Sci. USA 83:2511-2515)).

In addition, expression of the transgene can be precisely regulated, for example, by using an inducible regulatory sequence and expression systems such as a regulatory sequence that is sensitive to certain physiological regulators, e.g., circulating glucose levels, or hormones (Docherty et al., 1994, FASEB J. 8:20-24). Such inducible expression systems, suitable for the control of transgene expression in cells or in mammals include regulation by ecdysone, by estrogen, progesterone, tetracycline, chemical inducers of dimerization, and isopropyl-beta-Dl-thiogalactopyranoside (EPTG). A person skilled in the art would be able to choose the appropriate regulatory/promoter sequence based on the intended use of the dsRNA transgene.

In some embodiments, recombinant vectors capable of expressing oligonucleotides (e.g., siRNAs) molecules are delivered as provided herein and persist in target cells. In other embodiments, viral vectors can be used that provide for transient expression of oligonucleotides (e.g., siRNAs). In such cases, vectors can be repeatedly administered as necessary. Once expressed, the oligonucleotides (e.g., siRNAs) bind to target RNA and modulate its function or expression.

In some embodiments, delivery of oligonucleotides (e.g., siRNAs) expressing vectors can be systemic, such as by intravenous or intramuscular administration, by administration to target cells ex-planted from the patient followed by reintroduction into the patient, or by any other means that allows for introduction into a desired target cell. Successful introduction of vectors into host cells can be monitored using various known methods. For example, introduction can be signaled with a reporter, such as a fluorescent marker, such as Green Fluorescent Protein (GFP).

In some embodiments, the oligonucleotides (e.g., siRNAs) are administered to patient or a contacted with a cardiomyocyte using exosome that include the oligonucleotide-based therapeutic, for example, as described in U.S. Patent Publication No. 2020/0308587, which is herein incorporated by reference in its entirety.

### 5.4 Methods of Treating Heart Dysfunctions Using the Oligonucleotides and their Compositions

In another aspect, the disclosure features methods of treating cardiac dysfunction. In certain embodiments, the oligonucleotides have been developed based on parameters of potential to prevent or treat diastolic dysfunction. The methods can comprise administering to a subject in the need thereof a therapeutically effective amount of an oligonucleotide targeting Titin isoform N2B. In one embodiment, the present disclosure refers to an oligonucleotide inhibitor of human Titin N2B isoform that targets a nucleotide sequence present in Titin isoform N2B mRNA but not present in Titin pre-mRNA or Titin isoform N2BA mRNA. The inhibitor is preferably a nucleic acid molecule, including RNA molecules, DNA molecules, and modified nucleic acid molecules comprising at least one modified nucleic acid building block. In certain embodiments, the oligonucleotide can be delivered to cardiomyocytes of the subject.

In certain embodiments, the oligonucleotide can inhibit Titin isoform N2B by, for example, forming a double-stranded hybrid with the target. Additionally, the oligonucleotide can increase the ratio of Titin isoform N2BA (protein and/or mRNA) to Titin isoform N2B (protein and/or mRNA) and improve diastolic function in patients with diastolic heart failure (DHF). In certain embodiments, the oligonucleotide can suppress the expression of the Titin isoform N2B by about 2-fold to about 1000-fold greater than the oligonucleotide suppresses expression of the Titin isoform N2BA. In certain embodiments, the ratio of the Titin isoform N2BA to the Titin isoform N2B can be increased. In certain embodiments, the ratio of the Titin isoform N2BA to the Titin isoform N2B can be increased by about 1.2-fold to about 5-fold, such as by 1.2-fold, 1.5-fold, 1.8-fold, 2-fold, 2.2-fold, 2.5-fold, 2.8-fold, 3-fold, 3.5-fold, 4-fold, 4.5-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold or +/−10%.

In certain embodiments, the oligonucleotide can reduce expression of the N2B isoform of Titin by about 10% to about 90% (e.g., about 10% to about 80%, about 10% to about 70%, about 10% to about 60%, about 10% to about 50%, about 10% to about 40%, about 10% to about 30%, about 10% to about 20%, about 20% to about 90%, %, about 20% to about 80%, about 20% to about 70%, about 20% to about 60%, about 20% to about 50%, about 20% to about 40%, about 20% to about 30%, about 30% to about 90%, about 30% to about 80%, about 30% to about 70%, about 30% to about 60%, about 30% to about 50%, about 30% to about 40%, about 40% to about 90%, %, about 40% to about 80%, about 40% to about 70%, about 40% to about 60%, about 40% to about 50%, about 50% to about 90%, about 50% to about 80%, about 50% to about 60%, about 60% to about 90%, %, about 60% to about 80%, about 60% to about 70%, about 70% to about 90%, about 70% to about 80%, or about 80% to about 90%) as compared to a control (e.g., a human subject not administered an oligonucleotide that can reduce expression of the Titin isoform N2B). In some embodiments, the oligonucleotide can reduce expression of the N2B isoform of Titin by at least 10%, at least 20%, at least 30%, at least 40%, at least 50%, at least 60%, at least 70%, at least 80% or at least 90%+/−5%), as compared to a control (e.g., a human subject not administered an oligonucleotide that can reduce expression of the Titin isoform N2B).

In certain embodiments, the level of Titin isoform N2BA may not change appreciably upon administration of oligonucleotides to a cell. For example, it may be that the N2B isoform is targeted rather than the N2BA isoform. In such embodiments, the levels of the N2BA isoform may change by no more than 10%, no more than 8%, no more than 6%, no more than 4%, no more than 2%, or the N2BA levels may not detectably change (e.g., no more than 1%) in comparison to a cell to which the oligonucleotides have not been administered.

In certain embodiments, the composition of the present disclosure is useful in the prevention or treatment of cardiac disorders that exhibit diastolic dysfunction, particularly Heart Failure with preserved Ejection Fraction (HFpEF), Restrictive Cardiomyopathy, Hypertrophic Cardiomyopathy. In preferred embodiments, administration of the oligonucleotide improves diastolic dysfunction in the patient.

Additionally, the composition containing the oligonucleotide(s) can be delivered to one or more cells in vitro or ex vivo, (e.g., for the purposes of therapy or research). The Examples described herein show examples of such methods of the present disclosure. Any nucleotide of the present disclosure may be used therapeutically or in a research capacity.

### 5.5 Determining siRNA-Mediated Suppression of Expression

The ability of an oligonucleotide-containing composition of the present disclosure to inhibit protein synthesis can be measured using techniques which are known in the art, for example, by detecting an inhibition in gene transcription or protein synthesis. For example, Nuclease SI mapping can be performed. In another example, Northern blot analysis can be used to measure the presence of RNA encoding a particular protein. For example, total RNA can be prepared over a cesium chloride cushion (see, e.g., Ausebel et al., 1987. Current Protocols in Molecular Biology (Greene & Wiley, New York)). Northern blots can then be made using the RNA and probed (see, e.g., Id.). In another example, the level of the specific mRNA produced by the target protein can be measured, for example, using PCR. In yet another example, Western blots can be used to measure the amount of target protein present. Techniques for performing Western blots are well known in the art, see, e.g., Chen et al. J. Biol. Chem. 271:28259. In still another embodiment, a phenotype influenced by the amount of the protein can be detected. In another example, the promoter sequence of a target gene can be linked to a reporter gene and reporter gene transcription (e.g., as described in more detail below) can be monitored. Alternatively, oligonucleotide compositions that do not target a promoter can be identified by fusing a portion of the target nucleic acid molecule with a reporter gene so that the reporter gene is transcribed. By monitoring a change in the expression of the reporter gene in the presence of the oligonucleotide composition, it is possible to determine the effectiveness of the oligonucleotide composition in inhibiting the expression of the reporter gene. For example, in one embodiment, an effective oligonucleotide composition will reduce the expression of the reporter gene.

As used herein, “reporter gene” is a nucleic acid that expresses a detectable gene product, which may be RNA or protein. Detection of mRNA expression may be accomplished by Northern blotting and detection of protein may be accomplished by staining with antibodies specific to the protein. Preferred reporter genes produce a readily detectable product. A reporter gene may be operably linked with a regulatory DNA sequence such that detection of the reporter gene product provides a measure of the transcriptional activity of the regulatory sequence. In preferred embodiments, the gene product of the reporter gene is detected by an intrinsic activity associated with that product. For instance, the reporter gene may encode a gene product that, by enzymatic activity, gives rise to a detectable signal based on color, fluorescence, or luminescence. Examples of reporter genes include, but are not limited to, those coding for chloramphenicol acetyl transferase (CAT), luciferase, beta-galactosidase, and alkaline phosphatase.

One skilled in the art would readily recognize numerous reporter genes suitable for use according to the present disclosure. These include, but are not limited to, chloramphenicol acetyltransferase (CAT), luciferase, human growth hormone (hGH), and beta-galactosidase. Examples of such reporter genes can be found in F. A. Ausubel et al., Eds., Current Protocols in Molecular Biology, John Wiley & Sons, New York, (1989). Any gene that encodes a detectable product, e.g., any product having detectable enzymatic activity or against which a specific antibody can be raised, can be used as a reporter gene in the present methods.

One reporter gene system is the firefly luciferase reporter system. (Gould, S. J., and Subramani, S. 1988. Anal. Biochem., 7:404-408 incorporated herein by reference). In this assay, a lysate of the test cell is prepared and combined with ATP and the substrate luciferin. The encoded enzyme luciferase catalyzes a rapid, ATP dependent oxidation of the substrate to generate a light-emitting product. The total light output is measured and is proportional to the amount of luciferase present over a wide range of enzyme concentrations.

### 5.6 Kits

This disclosure also features kits comprising an oligonucleotide (e.g., any siRNA (e.g., sdRNA) provided herein) and that can be used to perform any of the methods described herein.

In certain embodiments, a kit comprises at least one oligonucleotide (e.g., siRNA) in one or more containers. In some embodiments, the kits contain all of the components necessary and/or sufficient to perform a detection assay, including all controls, directions for performing assays, and any necessary software for analysis and presentation of results. One skilled in the art will readily recognize that the disclosed oligonucleotide-based therapeutic can be readily incorporated into one of the established kit formats which are well known in the art.

### 6. EXAMPLES

Following are examples that illustrate procedures for practicing the present disclosure. These examples should not be construed as limiting. All percentages are by weight and all solvent mixture proportions are by volume unless otherwise noted.

### 6.1 Cell Culture Studies

Hela cells (ATCX, #CCL-2) were maintained in culture according to methods previously described (Chai et al., 1999) (Matthew R. Hassler et. al., Comparison of partially and fully chemically-modified siRNA oin conjugate-mediated delivery in vivo, Nucleic Acids Research, 2018). Plasmids were transiently transfected with Lipofectamine Plus (Invitrogen) in 12-well plates with cells plated at 70-90% confluency. For siRNA experiments, 1 μM of siRNA was transfected into cells cultured in antibiotic-free EMEM medium and 5% Fetal Bovine Serum. Cells and siRNAs were incubated for 48 hours before analysis.

### 6.2 Luciferase Assay

Luciferase assay was conducted as a duel-luc assay as previously described ((Matthew R. Hassler et. al., Comparison of partially and fully chemically-modified siRNA oin conjugate-mediated delivery in vivo, Nucleic Acids Research, 2018). Knockdown was measured using Renilla luciferase expression normalized to Firefly luciferase expression.

### 6.3 Example 1. Identifying of siRNA Targeting Human Titin Isoform N2B

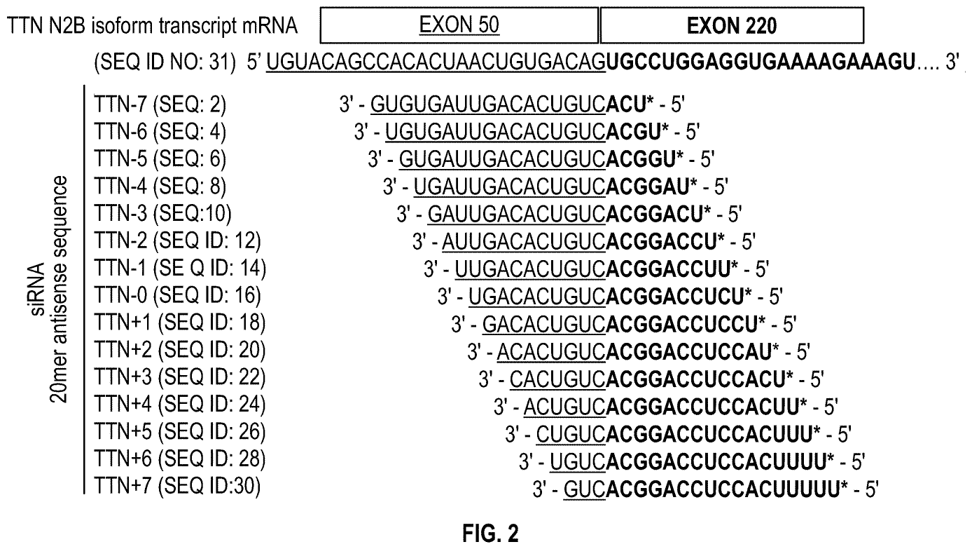

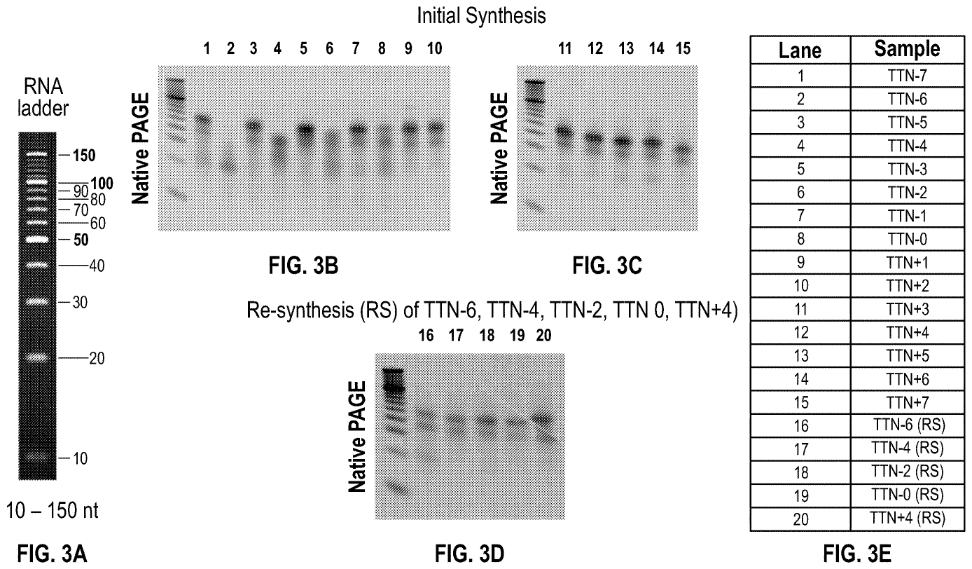

For the identification of the active siRNAs (e.g., sdRNAs) against human TTN N2B isoform (and/or Novex-1 and/or Novex-2), with little to no targeting of TTN N2BA isoform mRNA, 14 candidates centered around the “exon-exon boundary sequence” that is generated upon the splicing of TTN Exon 50 and exon 220 were synthesized as described in U.S. Pat. No. 10,933,081 and Shmushkovich et al., Nucleic Acids Res 46: 10905-10916 (2018), each of which are herein incorporated by reference in their entireties. The designed sequences are listed in FIG. 2 (and the sense and antisense sequences shown in Table 2). Antisense sequences as shown in FIG. 2 are designed with a uracil (U). For the primary screening, sdRNAs were synthesized as separate guide (antisense) and passenger (sense) strands (TriLink Biotechnologies; San Diego, CA) and dissolved in sterile RNase-free, DNase-free water for injection (CalBiochem, 4.86505) at 100 μM concentration and annealed by heating to 95° C. and gradually cooling to room temperature. FIGS. 3B-3E depict annealed duplexes were analyzed in the native gel electrophoresis. Duplexes were mixed with 5×TBE high-density sample buffer (Novex) and loaded in the TBE 4-20% gradient gels at 10 μmol per lane. Samples were fractionated at 150V and stained with SybrGold dye (ThermoFisher) for 10 min at RT. As a reference (M), we used 10 nucleotide (nt)-100 nt Low Molecular Weight Marker (Affimetrix) (FIG. 3A). Duplexes were formed in all the samples except for TTN−6, TTN−4, TTN−2 (FIGS. 3B-3C). Re-synthesis (RS) of TTN−6, TTN−4, TTN−2, TTN−0, and TTN+4 resulted in duplex formation (FIG. 3D).

Testing of siRNA complexes targeting human TTN N2B isoform (TNN−7, TTN−5, TTN−3, TTN−1, TTN−0, TTN+1, TTN+2, TTN+3, TTN+4, TTN+5, TTN+6, TTN+7) was performed. Native gel electrophoresis for TTN complexes was performed, where compounds were dissolved in sterile Rnase-, Dnase-, free water to the final concentration of 100 mM. The presence of the compounds was confirmed in the gel electrophoresis of FIGS. 3A-3E.

The siRNA solutions were stored at −80° C. Prior to use, the sdRNA stock solution was heated to 37° C. for 5 minutes, vortexed, and briefly spun down.

Self-deliverable siRNA (sdRNA) directed against TTN isoform N2B sequence, spanning TTN exon 50 and exon 220, shown in FIG. 2. were provided. These self-deliverable siRNAs were fully chemically modified siRNA-cholesterol conjugates. The modifications (f=2′ Fluro; m=2′ O-methyl; Ps=Phosphorothioate linkage) to the individual sequence are characterized as previously noted (see above).

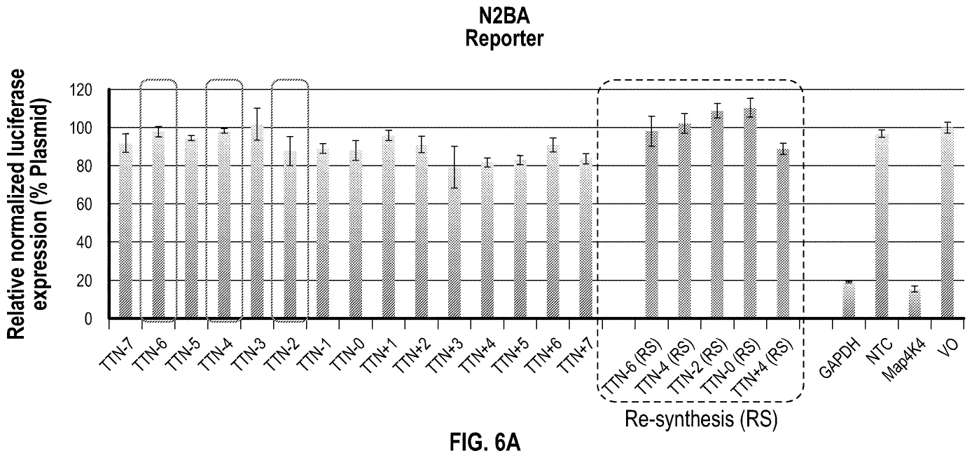

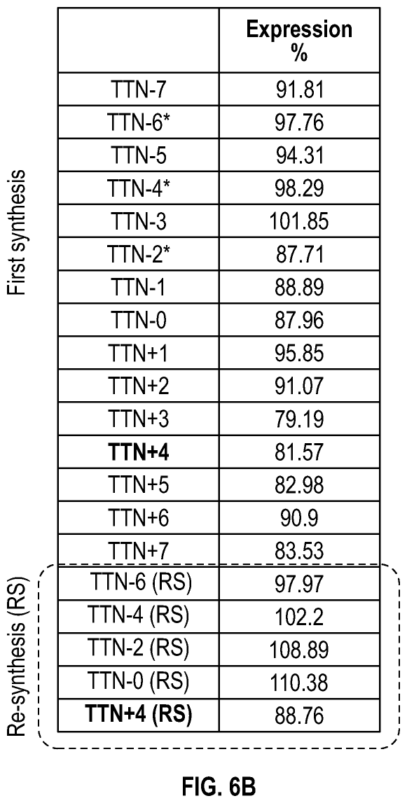

Reporter screening of TTN sdRNA compounds of the present disclosure is depicted in FIGS. 6A-6B, FIGS. 7A-7B, and FIG. 8.

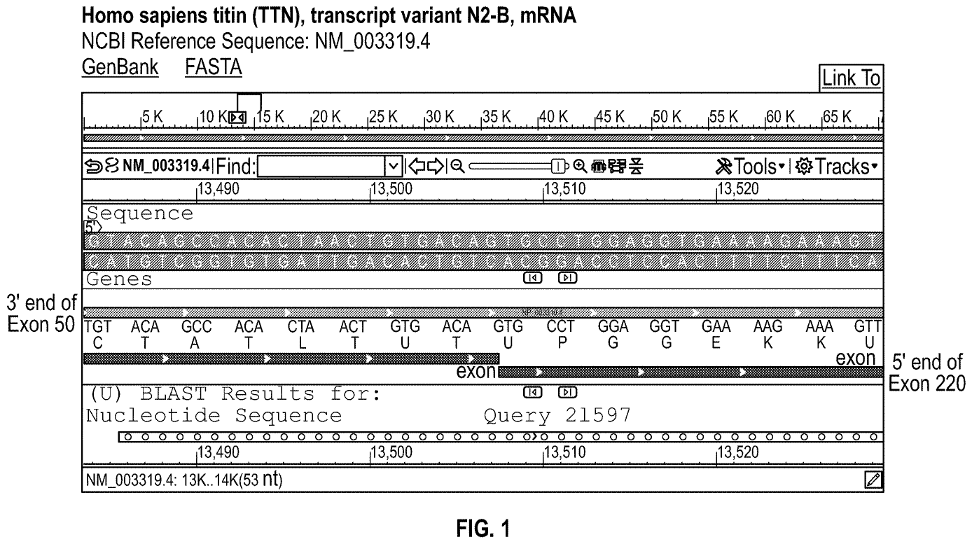

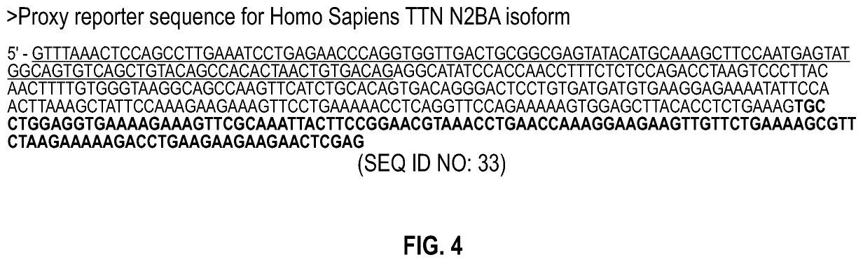

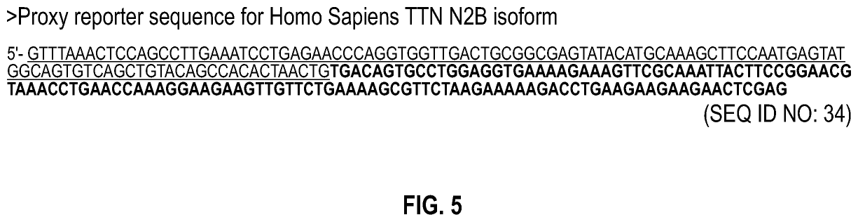

In particular, two dual luciferase plasmid reporters were generated to evaluate the ability of TTN sdRNAs to achieve targeted mediated suppression of TTN isoform N2B (or Novex-1 or Novex-2) with little to no effect on TTN N2BA isoform. The dual luciferase plasmid reporter that functioned as a proxy to evaluate target mediated suppression of TTN N2BA isoform contained the sequence (SEQ ID NO: 33) depicted in FIG. 4. This sequence contained the 5′ end of Exon 50, followed by a non-specific nucleotide sequence, followed by the 3′ end of Exon 220. The dual luciferase plasmid reporter that functioned as a proxy to evaluate target mediated suppression of TTN N2B isoform (or Novex-1 or Novex-2) mRNA contained the sequence (SEQ ID NO: 34) depicted in FIG. 5. This sequence contained the 5′ end of TTN exon 50 (depicted in FIG. 1) followed by the 3′ end of Exon 220 (depicted in FIG. 1). The TTN N2B isoform proxy reporter does not have a non-specific nucleotide sequence separating the 3′ end of Exon 50 and the 5′ end of Exon 220.

The reporting screening was performed under the following conditions: cell seeding: 10,000 Hela cells/well; 15 TTN sdRNAs were passively transfected into Hela cells expressing luciferase reporter; transfection: 1 μM compounds, antibiotic-free EMEM medium 3% Fetal Bovine Serum, 72 hour incubation. Knockdown was measured via Renilla luciferase expression and normalized to constant Firefly luciferase expression. Data is expressed as the percentage of gene expression of NTC transfected cells (NTC).

FIGS. 6A-6B show data for HeLa cells treated with 1.0 μM of TTN−7, TTN−6, TTN−5, TTN−4, TTN−3, TTN−2, TTN−2, TTN−1, TTN−0, TTN+1, TTN+2, TTN+3, TTN+4, TTN+5, TTN+6, TTN+7, or NTC and incubated in supplemented serum-free media for 72 hours. Data is reported as relative expression of TTN N2BA isoform 10/GAPDH+/−SD.

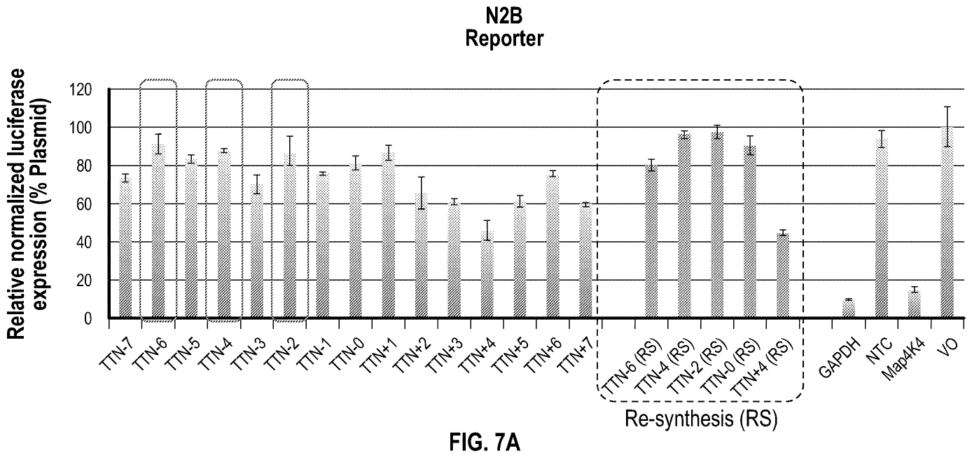

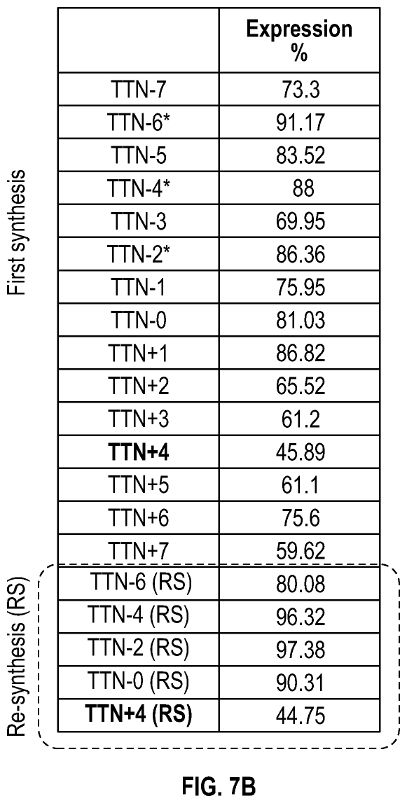

FIGS. 7A-7B show data for HeLa cells treated with 1.0 μM of TTN−7, TTN−6, TTN−5, TTN−4, TTN−3, TTN−2, TTN−2, TTN−1, TTN−0, TTN+1, TTN+2, TTN+3, TTN+4, TTN+5, TTN+6, TTN+7, or NTC and incubated in supplemented serum-free media for 72 hours. Data is reported as relative expression of TTN N2B isoform 10/GAPDH+/−SD is reported.

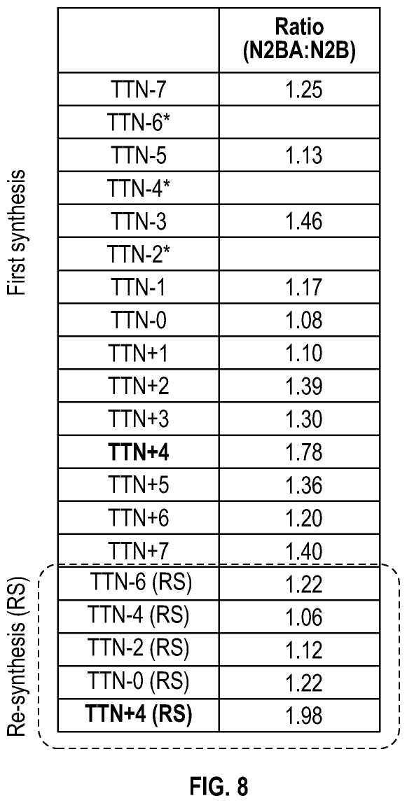

Overall, this data showed that not all siRNA (e.g., sdRNA) targeting TTN isoform N2B produced the same suppressive effect on expression. Surprisingly, siRNA consisting of an antisense strand having a sequence of either SEQ ID NO: 14 or SEQ ID NO: 16 (i.e., TTN−0 and TTN+1, respectively, in FIGS. 6A-6B and in FIGS. 7A-7B) did not significantly alter the ratio of Titin isoform N2BA to Titin isoform N2B (FIG. 8). In contrast, siRNAs with an antisense strand having a sequence selected from SEQ ID NOs: 20, 22, 24, 26, 28, and 30, each significantly increased the ratio of Titin isoform N2BA to Titin isoform N2B (see TTN+2, TTN+3, TTN+4/TTN+4 (RS), TTN+5, TTN+6, and TTN+7, respectively, in FIGS. 6A-6B, FIGS. 7A-7B, and FIG. 8). This result is particularly noticeable for the siRNAs with an antisense strand having a sequence selected from SEQ ID NOs: 22, 24, and 26 (TTN+3, TTN+4/TTN+4 (RS), and TTN+5, respectively), particularly SEQ ID NO: 24 (TTN+4/TTN+4 (RS)). This data provides proof-of-principle that siRNA targeting TTN isoform N2B can increase the ratio of Titin isoform N2BA to Titin isoform N2B and, therefore, can improve diastolic function in patients with diastolic heart failure (DHF).

### 7. EQUIVALENTS AND INCORPORATION BY REFERENCE

All references cited herein are incorporated by reference to the same extent as if each individual publication, database entry (e.g., Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. In particular, U.S. Provisional Patent Application No. 63/384,389, filed Nov. 18, 2022, is hereby incorporated by reference in its entirety.

While aspects of the present disclosure have been particularly shown and described with reference to preferred embodiments and various alternate embodiments, it will be understood by persons skilled in the relevant art that various changes in form and details can be made therein without departing from the spirit and scope of the present disclosure.

1. A method of treating or preventing diastolic dysfunction in a human subject, comprising administering to the subject an oligonucleotide that suppresses the expression of the Titin isoform N2B.

2-6. (canceled)

7. The method of any one of claim 1, wherein the oligonucleotide is double-stranded.

8. The method of any one of claim 1, wherein the oligonucleotide is a small interfering RNA (siRNA), a short hairpin RNA (shRNA), or a micro RNA (miRNA).

9. The method of any one of claim 1, wherein the oligonucleotide comprises a small interfering RNA (siRNA).

10. The method of claim 9, wherein the siRNA comprises (a) a duplex region and (b) optionally one or two overhang regions, wherein each overhang region is six or fewer nucleotides, wherein the duplex region comprises a sense and an antisense region each comprising between 15 and 30 nucleotides, and wherein the antisense region comprises a nucleotide sequence that is substantially complementary to SEQ ID NOs: 31 or 32.

11. The method of claim 10, wherein the antisense region comprises a nucleotide sequence that is substantially complementary to a contiguous sequence of at least 18 nucleotides of SEQ ID NO: 31.

12. The method of claim 11, wherein the antisense region is 85% complementary to a contiguous sequence of at least 18 nucleotides of SEQ ID NO: 31.

13. The method of claim 11, wherein the antisense region is 90% complementary to a contiguous sequence of at least 18 nucleotides of SEQ ID NO: 31.

14. The method of claim 11, wherein the antisense region is 95% complementary to a contiguous sequence of at least 18 nucleotides of SEQ ID NO: 31.

15. The method of claim 11, wherein the antisense region is 100% complementary to a contiguous sequence of at least 18 nucleotides of SEQ ID NO: 31.

16. The method of any one of claim 11, wherein the contiguous sequence comprises positions 7 to 24 of SEQ ID NO: 31.

17. The method of any one of claim 11, wherein the contiguous sequence comprises positions 6 to 23 of SEQ ID NO: 31.

18. The method of any one of claim 11, wherein the contiguous sequence comprises positions 8 to 25 of SEQ ID NO: 31.

19. The method of claim 10, wherein the antisense region comprises a nucleotide sequence that is substantially complementary to SEQ ID NO: 32.

20. The method of claim 19, wherein the antisense region is at least 85% complementary to SEQ ID NO: 32.

21. The method of claim 19, wherein the antisense region is at least 90% complementary to SEQ ID NO: 32.

22. The method of claim 19, wherein the antisense region is at least 95% complementary to SEQ ID NO: 32.

23. The method of claim 19, wherein the antisense region is 100% complementary to SEQ ID NO: 32.

24. The method of any one of claim 19, wherein the sense and antisense regions each comprise between 13 and 25 nucleotides.

25. The method of any one of claim 19, wherein the sense and antisense regions each comprise between 15 and 22 nucleotides.

26. The method of any one of claim 9, wherein the antisense sequence has at least 85% sequence identity to a sequence selected from SEQ ID NOs: 2, 4, 6, 8, 10, 12, 14, 22, 24, 26, 28, and 30.

27. The method of any one of claim 9, wherein the antisense region has at least 90% complementary to a sequence selected from SEQ ID NOs: 2, 4, 6, 8, 10, 12, 14, 22, 24, 26, 28, and 30.

28. The method of any one of claim 9, wherein the antisense region has at least 95% sequence identity to a sequence selected from SEQ ID NOs: 2, 4, 6, 8, 10, 12, 14, 22, 24, 26, 28, and 30.

29. The method of any one of claim 9, wherein the antisense region is a sequence selected from SEQ ID NOs: 2, 4, 6, 8, 10, 12, 14, 22, 24, 26, 28, and 30.

30-173. (canceled)
